(19) United States
(12) Patent Application Publication
(10) Pub. No.: US 2011/0229928 A1
(43) Pub. Date: Sep. 22, 2011

(54) DEVICE AND METHOD FOR MICROWAVE ASSISTED CRYO-SAMPLE PROCESSING

(76) Inventors: David W. Dorward, Hamilton, MT (US); Vinod Nair, Hamilton, MT (US); Elizabeth Fischer, Hamilton, MT (US); Bryan Hansen, Hamilton, MT (US)

(21) Appl. No.: 13/062,479

(22) PCT Filed: Sep. 3, 2009
(86) PCT No.: PCT/US2009/055913

§ 371 Date: May 25, 2011

Related U.S. Application Data
(60) Provisional application No. 61/094,848, filed on Sep. 5, 2008.
(60) Provisional application No. 61/112,575, filed on Nov. 7, 2008.

(51) Int. Cl. C12Q 1/02; C12M 1/00; C12M 3/00

(52) U.S. Cl. 435/29; 435/283.1

(57) ABSTRACT

Embodiments are provided that provide for devices and methods for microwave-assisted cryo-sample processing. In some embodiments, a system for microwave-assisted cryo-sample processing of a sample includes a chamber adapted to receive microwave radiation and a device disposed in the chamber that is configured to maintain a sample under cryo conditions during irradiation of the sample with microwave radiation.

### FIELD OF THE INVENTION

The present disclosure relates to devices and methods for microwave-assisted cryo-sample processing.

### BACKGROUND

Freeze substitution of rapidly frozen hydrated samples with fixatives dissolved in organic solvents is commonly used for preservation and accurate representation of microscopic structure and ultrastructure. Grahamm, L. L., and Beveridge, T. J., J. Bacteriol. 176: 1413-21 (1994); Leapman, R. D, et al., Ultramicroscopy 100: 115-25 (2004); Leapman, R. D., Curr. Opin. Neurobiol., 14: 591-8 (2004); Lucic, V. et al., Ann. Rev. Biochem. 74: 833-65 (2005); Matias, V. R. F., and Beveridge, T. J., Mol. Microbiol. 64: 195-206 (2007); Matias, V. R. F., and Beveridge, T. J., Mol. Microbiol. 56: 240-51 (2006), McDonald, K. L., Auer, M., Biotechniques 41:137-9 (2006). Freeze substitution is the process of dissolving ice and freezing medium components in a frozen specimen by an organic solvent at low temperature and usually takes place in the presence of a secondary fixative. Steinbrecht and Muller, Cryotechniques in Biological Electron Microscopy, Steinbrecht and Zierold (Eds). Berlin:Springer-Verlag, pp. 149-172 (1987). In contrast to relatively slow inactivation of cellular components that occurs by diffusion of chemical fixatives, rapid freezing immobilizes and inactivates living cells in milliseconds or less. Furthermore, small aqueous solutes in hydrated materials fixed by immersion and diffusion of chemical cross-linkers are typically extracted from samples during repeated fluid exchanges.

During freeze substitution, low-temperature substitution of dehydrating agents and fixatives into rapidly frozen samples allows for the crosslinking of cellular components and the removal of water at temperatures low enough to avoid the damaging effects of ambient temperature dehydration. In addition, freeze substitution fixation retains many such hydrophilic solutes facilitating later detection and quantification by x-ray and electron energy loss microanalysis. Similarly, reactivity of antigens in samples is also preserved by freeze substitution more frequently than by chemical fixation, primarily by processing at low temperature often without the use of bi-functional protein cross-linking agents such as glutaraldehyde or carbodiimides. Lucic, V. et al., Ann. Rev. Biochem. 74: 833-65 (2005); McDonald, K. L. and Auer, M., Biotechniques 41: 137-9 (2006); Ohno, N., et al., Histol. Histopathol. 22: 1281-90 (2007); Saitoh, et al., J. Immunol. Methods 331: 114-26 (2008); Schwartz, C. I., et al., J Microscopy 227: 98-109 (2007). However, despite its advantages, routine use of freeze substitution has been limited in clinical and research settings in large part due to the extended time periods required for processing, which involves passive diffusion of organic solutions into frozen material at cryo temperatures.

### SUMMARY OF THE INVENTION

The present teachings provide, among other things, systems, devices and methods that facilitate processing of samples under cryo conditions.

Various embodiments of a system of the present teachings comprise: a chamber adapted to receive microwave radiation; and a cooling/heating device disposed in the chamber, wherein the cooling/heating device is configured to maintain a sample under cryo conditions during irradiation of the sample with microwave radiation.

In some embodiments, the cooling/heating device is adapted to conduct a cryogenic substance therethrough.

In some embodiments, the system further comprises a sample holder comprising at least one well, wherein the well is configured to receive the sample, and wherein the sample holder is configured to be disposed in a recess in the cooling/heating device.

In some embodiments, the system further comprises a temperature sensor. In some embodiments, the cooling/heating device can be configured to maintain the temperature of the sample between about −200° C. and about +20° C. (e.g., at least, greater than, less than, or equal to about −200, −150, −100° C., −90° C., −80° C., −70° C., −60° C., −50° C., −40° C., −30° C., −20° C., −10° C., 0° C., 10° C., or 20° C.). In some embodiments, the system further comprises a temperature regulation system operably connected to the cooling/heating device.

In some embodiments, the system further comprises a programmable controller. The controller can be programmed with a temperature setting and/or a microwave setting (e.g., frequency, wavelength, time of irradiation, oscillation of frequency or wavelength).

In some embodiments, the system further comprises a venting system for removing vapors from the sample. In some embodiments, the system further comprises a vacuum system for regulating sample pressure. In some embodiments, the system further comprises a dry-gas purge system for reducing moisture in the chamber. In some embodiments, the system is configured from materials that are compatible within the range of temperatures of between about −200° C. and about +20° C. (e.g., at least, greater than, less than, or equal to about −200, −150, −100° C., −90° C., −80° C., −70° C., −60° C., −50° C., −40° C., −30° C., −20° C., −10° C., 0° C., 10° C., or 20° C.) in the presence and absence of microwave irradiation and chemical exposure.

In some embodiments, the system further comprises one or more microwave sources. Optionally, the microwave source is operably connected to the sample holder comprising at least one well, wherein the well is configured to receive the sample, such that the sample holder is configured to be disposed in a recess in the cooling/heating device while receiving microwave irradiation. Preferably, the microwave generating device or microwave source is device that is configured to produce RF waves in approximately the 2.45 GHz range; however, some embodiments comprise microwave generating devices that are configured produce RF waves that are at least, equal to greater than or less than 900 MHz to 25 GHz (e.g., at least, equal to greater than or less than about 900 MHz, 1 GHz, 1.2 GHz, 1.5 GHz, 1.8 GHz, 2.0 GHz, 2.2 GHz, 2.4 GHz, 3.6 GHz, 2.8 GHz, 3.0 GHz, 3.5 GHz, 4.0 GHz, 4.5 GHz, 5.0 GHz, 5.5 GHz, 6.0 GHz, 7.0 GHz, 8.0 GHz, 9.0 GHz, 10.0 GHz, 11.0 GHz, 12.0 GHz, 13.0 GHz, 14.0 GHz, 15.0 GHz, 16.0 GHz, 17.0 GHz, 18.0 GHz, 19.0 GHz, 20.0 GHz, 21.0 GHz, 22.0 GHz, 23.0 GHz, 24.0 GHz, or 25 GHz). In some embodiments, the microwave source is a magnetron and in other embodiments, the microwave source is a plasma electromagnetic generator and in more embodiments, the microwave source is a semiconductor diode or triode (e.g., a Gunn-diode oscillator or tunnel diode). In some embodiments the system further comprises an oscillator coupled to the microwave source or configured to pulse the sample with microwave irradiation in a repetitive fashion (e.g., timed pulses of a set or variable frequencies coordinated with a temperature regulator so as to maintain the cryo environment). In some embodiments, the system further comprises a microwave attenuator, which is configured to control the amount of radiation entering into the sample chamber and/or coming into contact with the sample. Examples of such microwave attenuators include filters, shutters, electromagnetic field compensators, wave canceling devices, and wave jamming devices. In some embodiments, the microwave attenuator is regulated by a temperature sensor and/or a user defined input such that once a user defined threshold temperature in the sample chamber is reached, the microwave attenuator is engaged. The microwave attenuator can be attached to the sample chamber and oriented such that it blocks the sample from receiving the microwave radiation when it is engaged or the microwave attenuator can be attached to the sample holder and oriented such that it blocks the sample from receiving microwave radiation once it is engaged. In some embodiments, the system further comprises a heat sink or thermal dispersion device configured to evenly distribute heat (e.g., ColdSpot™).

In some embodiments a chemical composition is disposed within the chamber, wherein the chemical composition is in contact with the sample.

In some embodiments, the system further is configured such that the sample is substantially impregnated by the chemical composition in less than about two hours. In some embodiments, the system further is configured such that the sample is substantially impregnated by the chemical composition in less than about twenty minutes.

Various embodiments of a cooling/heating device of the present teachings comprise: a block comprising at least one opening sized to fit one or more samples, wherein the block is translucent or opaque to microwave irradiation and adapted to contain or conduct a cryogenic substance therethrough, and wherein a sample held by the block is maintained under cryo conditions during microwave irradiation.

In some embodiments, the cooling/heating device further comprises one or more temperature sensors, which may be configured to regulate the pulses of microwave radiation emitted from the microwave source and/or engagement of the microwave attenuator. That is, the temperature sensor may be configured such that once a user defined threshold temperature in the sample chamber is reached, the microwave irradiation is stopped or attenuated or deflected from the sample.

Various embodiments of a sample holder of the present teachings comprise: a microwave-translucent or opaque container comprising at least one opening configured to hold one or more samples, wherein the samples held in the sample holder are oriented for uniform microwave irradiation. In some embodiments, the microwave attenuator is attached to the sample holder.

Various embodiments of a method of the present teachings comprise: irradiating a sample with a first power microwave radiation for a first set time, wherein the sample is maintained under cryo conditions and does not thaw during the first set time. That is, some embodiments comprise a microwave generating device that are configured to irradiate a sample with at least, equal to greater than or less than about 900 MHz to 25 GHz (e.g., at least, equal to greater than or less than about 900 MHz, 1 GHz, 1.2 GHz, 1.5 GHz, 1.8 GHz, 2.0 GHz, 2.2 GHz, 2.4 GHz, 3.6 GHz, 2.8 GHz, 3.0 GHz, 3.5 GHz, 4.0 GHz, 4.5 GHz, 5.0 GHz, 5.5 GHz, 6.0 GHz, 7.0 GHz, 8.0 GHz, 9.0 GHz, 10.0 GHz, 11.0 GHz, 12.0 GHz, 13.0 GHz, 14.0 GHz, 15.0 GHz, 16.0 GHz, 17.0 GHz, 18.0 GHz, 19.0 GHz, 20.0 GHz, 21.0 GHz, 22.0 GHz, 23.0 GHz, 24.0 GHz, or 25 GHz for at least, greater than, less than or equal to about 1 to 300 seconds (e.g., at least, equal to greater than or less than about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 50, 60, 70, 80, 90, 100, 140, 180, 200, 220, 240, 280, or 300 seconds) while maintaining a temperature of between about −200° C. and about +20° C. (e.g., at least, greater than, less than, or equal to about −200, −150, −100° C., −90° C., −80° C., −70° C., −60° C., −50° C., −40° C., −30° C., −20° C., −10° C., 0° C., 10° C., or 20° C.) in the presence or absence of one or more chemicals including but not limited to acetone, methanol, ethanol, O_{S}O₄ (osmium tetroxide) uranyl acetate, tannic acid, glutaraldehyde, paraformaldehyde, ruthenium tetroxide, picric acid, ruthenium red, alcian blue, potassium permanganate, or a carbodimide.

In some embodiments, the method further comprises irradiating the sample with a second power microwave radiation for a second set time. In some embodiments, the method can include additional power settings and additional corresponding time periods. In some embodiments, the microwave irradiation is pulsed on and off for set time periods or is applied to the sample and then attenuated in a repetitive fashion while maintaining a constant or an about constant temperature (e.g., +/−1, 2, 3, 4, 5, 6, 7, 8, 9, 10, 11, 12, 13, 14, or 15° C.).

In some embodiments, the method further comprises contacting the sample with a first chemical composition during the first set time. In some embodiments, the chemical composition comprises an organic solvent selected from the group consisting of acetone, methanol and ethanol. In some embodiments, the chemical composition comprises an inorganic solvent. In some embodiments, the chemical composition comprises one or more compounds such as, for example, O_{S}O₄ (osmium tetroxide), uranyl acetate, tannic acid, glutaraldehyde, paraformaldehyde, ruthenium tetroxide, picric acid, ruthenium red, alcian blue, potassium permanganate, and/or a carbodimide.

### BRIEF DESCRIPTION OF THE DRAWINGS

FIGS. 1A-B depict a schematic illustration of a microwave-assisted cryo-sample processing system. (A) Exemplary cryo-sample processing system. (B) Exemplary sample holder.

FIG. 2 depicts one embodiment of a sample holder for microwave-assisted cryo-sample processing.

FIG. 3 depicts a schematic illustration of one embodiment of a cooling/heating device sample holder.

FIG. 4 depicts a schematic illustration of one embodiment of a sample holder.

FIG. 5 depicts a schematic illustration of system including a sample holder adapted to fit in a cooling/heating device.

FIGS. 6A-F depict images of Bacillus subtilis sections prepared by (A) conventional fixation, (B) ambient microwave (MW)-assisted chemical fixation, (C) traditional passive freeze substitution fixation (FS), (D) rapid FS without MW irradiation, (E) rapid FS with MW irradiation, (F) rapid FS with MW, higher magnification.

FIGS. 7A-B depict immuno-electron microscopic images of sections from merozoites from Plasmodium falciparum-infected erythrocytes prepared by rapid FS with MW irradiation. (A). (B) Section prepared by rapid FS with MW irradiation labeled with anti-PfM6Tα primary antibody, and colloidal gold-conjugated secondary antibody. I, inner membrane complex; P, merozoite plasma membrane; PVM, parasitophorous vacuolar membrane; R, rhoptry.

FIG. 8 depicts an automated freeze substitution device (AFS-2) configured to receive a microwave source, showing the liquid nitrogen (LN2) vessel and attachments to the sample chamber.

FIG. 9 depicts a sample chamber of an automated freeze substitution device (AFS-2) configured to receive a microwave source.

FIG. 10A-B depicts a top-side view (A) and bottom view (B) of a microwave source configured for integration with an automated freeze substitution device (AFS-2).

FIG. 11 illustrates pre-fixed human HeLa cells that were initially fixed with 2% paraformaldehyde in 0.1 M cacodylate buffer pH 7.2, and 2.5% glutaraldehyde and 0.05% malachite green in cacodylate buffer, then transferred to membrane carriers and high-pressure frozen. Frozen samples were further processed by MWFS (microwave-assisted freeze-substitution) using sequentially administered reagents over approximately 4-6 hours. Panels A-D are sections of HeLa cells at various magnifications showing that excellent morphological and fine structural details can be preserved rapidly by MWFS. Bars: 100 nm.

FIG. 12A-F illustrates microwave assisted freeze substitution of Plasmodium falciparum in human red blood cells. Samples were prepared by ambient microwave-assisted chemical fixation (A) and (B), conventional freeze substitution (C) or (D), or MWFS (E) or (F). Examination of sections from samples prepared by each method demonstrated that MWFS processing resulted in superior overall morphology of the parasites. In particular, fine structures such as parasite membrane complexes were absent or poorly resolved by ambient (B) and traditional FS processing (D), yet well preserved and delineated by MWFS (F). Bars: A, C, E-500 nm, B, D, F-100 nm.

### DETAILED DESCRIPTION

Various embodiments described herein are directed towards systems, devices and methods maintaining samples at cryo-temperatures during microwave processing. In some embodiments, the systems, devices and methods can be used to accelerate freeze substitution fixation (FS). The systems, devices and methods described herein provide preservation favorably comparable to that achieved using conventional chemical fixation or traditional freeze substitution in a few hours rather than days.

FS of hydrated samples frozen in vitreous ice provides exceptional preservation of structure for light and electron microscopy. Furthermore, FS often enables immunological detection of thermo-labile antigens that otherwise are damaged or destroyed by processing at ambient or elevated temperatures. However, use of FS as a tool for research and clinical pathology has been hindered by relatively lengthy periods required for diffusion of fixatives and organic solvents into the frozen hydrated material. Standard FS generally takes several days, such as, for example, approximately 2 to 6 days. Protocols for traditional FS typically use multiple temperatures such as, for example, about −90° C., about −80° C., and about −40° C. to optimize incubation temperature with the reactivity of fixative reagents (Lucic, et al., Ann. Rev. Biochem. 74: 833-65, (2005); McDonald et al., Biotechniques 41: 137-9. (2006); Thirion et al., J. Microsc. 186: 28-34. 1997).

Controlled microwave irradiation dramatically shortens time periods required for light and electron microscope sample processing (Giberson, R. T., and Demaree, R. S. (2001); Munoz et al., J. Neurosci. Methods 137: 133-9 (2004); Schroeder, J. A., et al. Micron 378: 577-90 (2006); Webster, P. Methods. Mol. Biol. 369: 47-65 (2007). Similarly, the irradiation also promotes sample staining and immunogold and immune histochemical labeling procedures (Giberson (2001); Ohno (2007). Microwave ovens similar in design to those used in home cooking have been used to accelerate the time required for tissue processing. For example, U.S. Pat. No. 4,656,047 claims a method of tissue processing that utilizes microwave energy. U.S. Pat. No. 4,839,194 also describes a method of fixing a tissue where microwave energy is used. U.S. Pat. Nos. 4,839,194 and 5,244,787 described a method of staining tissue specimens utilizing microwave energy.

Some of the present embodiments involve systems and devices useful for microwave-assisted processing of frozen samples under cryo-conditions. As used herein “under cryo-conditions” refers to conditions under which samples remain frozen and do not thaw. For example, the devices can facilitate and enhance freeze substitution fixation of frozen materials. In some embodiments, the devices can automate, control, and maintain samples at cryo-temperatures during microwave processing. In some embodiments, microwave-assisted cryo-sample processing can be performed using, for example, existing clinical research laboratory microwave processors. In other embodiments, microwave-assisted cryo-sample processing can be carried out using standard or optional attachments to conventional microwave processors. In other embodiments, microwave-assisted cryo-sample processing can be carried out using a dedicated cryo-microwave processor. In other embodiments, a system for freeze substitution can be adapted for microwave-assisted cryo-sample processing. For example, a freeze substitution processor can be adapted for use in combination with a microwave source such as, for example, a magnetron. Examples of freeze substitution processors that can be adapted for microwave processing include but are not limited to, for example, the Leica™ EM AFS2 system and the Leica™ AFS system.

Some of the present embodiments involve methods for processing frozen samples in a laboratory MW processor under cryo-conditions. In some embodiments, freezing, processing, and infiltrating by microwave-assisted freeze substitution (MWFS) can be completed in less than about 4-6 hours, compared with approximately 5 days for standard FS. In some embodiments, microwave irradiation reduces the time period required for freeze substitution from approximately 2 to 6 days to about 2 to 3 hours. Thus, in some embodiments, microwave processing can facilitate and enhance FS of frozen material for light and electron microcopy and other purposes.

In some embodiments, devices for holding frozen samples during microwave-assisted cryo-sample processing are provided. Inconsistent microwave radiation exposure can lead to variable fixation of the samples, including incomplete fixation of some samples. For example, during microwave-assisted cryo-sample processing, the presence of multiple high-pressure freezing carriers, which are often metallic, and can be present in each vial or container being processed can result in the samples being inconsistently exposed to radiation. Sample holders for separating and orienting samples such that samples are reproducibly and uniformly fixed during microwave-assisted cryo-sample processing are provided. In some embodiments, a sample may or may not be removed from a high-pressure freezing carrier prior to placement in the sample holder. Separation and orientation of the samples by the holder ensures equivalent microwave dosage among samples within the microwave chamber. Thus, the holders prevent variable fixation among samples processed in the same vial or container. In some embodiments, the holders are configured to contain a cooling/heating medium, or have a medium circulated therethrough for regulating sample temperature. In some embodiments, the holders or containers are microfuge tubes, cryovials, or Beem® capsules. Some holders used with the methods and devices described herein are containers that are configured to accommodate large tissues, tissue biopsies, insects, plant tissue.

As will be appreciated by one of skill in the art, the ability to quickly prepare high quality frozen samples can have great benefit, especially for applications where rapid turnaround and high quality preservation is desirable. For example, microwave-assisted cryo-sample processing can be utilized for excellent preservation and rapid turnaround in research and high throughput clinical laboratory settings. The devices and methods disclosed herein are useful for a wide range of applications in, for example without limitation, light and electron microscopy for clinical facilities, forensics, biological research, biomedicine, biodefense, material fields (including hydrated-materials research), product development, production and quality control. In addition, the methods and devices disclosed herein are applicable for structural analyses of hydrogels that are not well preserved by traditional amine or carboxylic acid cross-linking reagents. Such hydrogels include, for example without limitation, biological and synthetic products such as polysaccharides, and commercial items such as contact lenses, prosthetic devices, Synvisc® (hylan), cheeses, other food products, paints, coatings, forensics products, cosmetics and many other liquids and emulsions in the food and material industries. The systems, devices and methods disclosed herein can also be used in a broad range of low-temperature chemical and biological procedures other than microwave-assisted cryo-sample processing such as, for example without limitation, rapid immunolabeling and embedding for histological preparations conducted below ambient temperatures, analytical and synthetic chemistry to speed reactions.

### Systems and Devices

Some of the present embodiments involve systems and devices useful for microwave-assisted processing of samples under cryo-conditions. In some embodiments, the systems can automate, control, and maintain samples at cryo-temperatures during microwave processing. To accomplish cryo-temperature control for samples processed with microwave irradiation, the systems include a chamber that receives microwave irradiation from a microwave source, and a device for regulating temperature of samples, such as, for example, a cooling/heating plate or block.

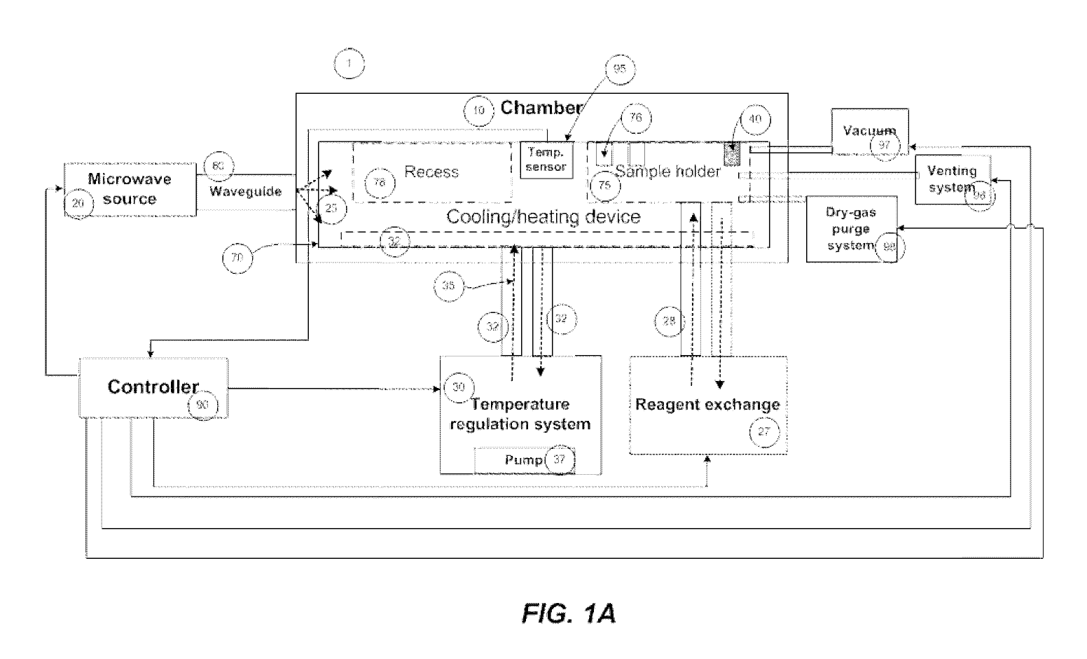

With reference now to FIG. 1A, an exemplary system 1 for microwave-assisted cryo-sample processing is illustrated. For applying microwave radiation, any suitable microwave source 20 can be used in conjunction with a chamber 10 adapted to receive microwave irradiation. In some embodiments, a microwave oven including a microwave source 20 and a chamber 10 can be used. In some embodiments, the microwave oven can be a laboratory microwave processing system. Various laboratory microwave processing systems are known in the art and can be adapted for various embodiments disclosed herein. Examples of laboratory microwave ovens are described at, for example, the Leica™ Microsystems, Ted Pella, Inc., Vibratome™, Microwave Research & Applications, Inc., Electron Microscopy Sciences, EBSciences, Hacker Instruments & Industries, Inc., and Triangle Biomedical Sciences websites, all of which provide examples of current laboratory microwave processing systems. One possible robotic microwave processor that can be adapted for various embodiments disclosed herein is the Leica™ EM AMW. In some embodiments, a waveguide 60 can be used to transmit the microwave energy 25 from the microwave source 20 to the chamber 10. Preferably, the microwave processing system is adapted with inlet and outlet ports to allow the attachment of, for example, temperature regulation systems, reagent exchange systems, vacuum pump, purge systems, venting systems, etc.

In some embodiments, one or more sample holders 75 can be used to separate and orient samples such that samples are reproducibly uniformly fixed during microwave-assisted cryo-sample processing are provided. The sample holder can hold one or more samples during microwave-assisted cryo-sample processing of the sample.

The temperature of the sample holder 75 can be adjusted to a desired temperature, such as a cryo-temperature or ambient temperature. In some embodiments, the sample holder can be placed into a cooling/heating device 70, which can regulate the temperature of the sample holder and samples therein. In other embodiments, the cooling/heating device itself can be configured to hold one or more samples. For example, the cooling/heating device can include one or more recesses, pores, wells or slots 78 configured to hold one or more samples and/or sample holders. In some embodiments, the cooling/heating device can function as a sample holder.

In some embodiments, the temperature of the system 1 for microwave-assisted cryo-sample processing can be regulated by a temperature regulation system 30. In some embodiments, the temperature regulation system can be external to the microwave oven. For example, the temperature regulation system can be an external manual or programmable external temperature regulation system. In other embodiments, the temperature regulation system 30 can be integrated with the microwave oven.

In other embodiments, a cooling/heating device disposed in the chamber can include a cooling/heating medium for regulating temperature. As used herein “cooling/heating medium” refers to a medium that can be used to regulate temperature, including cooling and heating. For example, media such as, for example without limitation, liquid or vaporous nitrogen, solvent(s) or refrigerant(s), cryogenic substances or other suitable substances can be added to the cooling/heating device for regulating temperature. In some embodiments, the substance can be non-polar.

In some embodiments, a circulation system can deliver a cooling/heating medium 35 to a cooling/heating device 70 disposed inside the microwave chamber such as, for example, a plate or block. For example, a cooling/heating device 70 disposed in the microwave chamber 10 can be configured to receive and circulate cooling/heating medium therethrough.

The cooling/heating device 70 can be used to regulate the temperature of one or more samples 40. In some embodiments, the cooling/heating device 70 is configured to hold one or more sample holders 75 containing one or more samples 40. For example, the cooling/heating device can include a recess 78 or opening that can fit a sample holder 75 holding a plurality of samples 40. In other embodiments, the cooling/heating device 70 can be a sample holder and is configured to directly receive one or more samples 40. For example, the cooling/heating device can include one or more recesses, pores, wells or slots 78 that can fit one or more samples 40 or sample holders 75. Samples holders are described in more detail below. In some embodiments, sample temperature can be regulated by regulating the sample holder 75 temperature. In other embodiments, the sample temperature can be regulated by regulating the temperature within the chamber 10.

In some embodiments, the temperature regulation system 30 is capable of generating and delivering gas or liquid at temperatures ranging from about −200° C. or below to about ambient temperature or greater. A cooling/heating medium 35 can be passed through the walls of the microwave chamber via lines or tubing 32. The lines and tubing can be constructed of any suitable material for carrying the cooling/heating medium and is not meant to be limited to any particular material. In some embodiments, the lines and tubing can be constructed of microwave-opaque material. In other embodiments, the lines and tubing can be constructed of microwave translucent material such as, for example without limitation, Teflon®.

A variety of media useful for regulating temperature are known and include, for example without limitation, liquid or vaporous nitrogen, solvent(s) or refrigerant(s), cryogenic substances or other suitable substances. In some embodiments, the medium can be a refrigerant that can operate at typical FS temperatures in compressor/adiabatic systems such as ultra-cold freezers. Examples of suitable media include, without limitation, fluorinated hydrocarbons such as, for example, R23, R508B, R503, R13, and others. In some embodiments, the cooling medium is non-toxic. In some embodiments, the cooling medium is non-polar. In some embodiments, the cooling medium can be inert. In some embodiments, a temperature regulation system can include, for example, thermoelectric cooling and heating.

Various temperature regulation systems are known in the art, and any suitable temperature regulation system can be used in the system 1 to adjust the temperature of the cooling/heating device 70 to desired temperatures, including, for example, cryo-temperatures. In some embodiments, the entire chamber 10 can be cooled to a desired temperature. In some embodiments, a temperature regulation system can include, for example without limitation, a heat pump or a heat exchanger, or combination thereof. The temperature regulation system can include, for example, an ultra-cold refrigeration compressor device or, for example, a liquid nitrogen cooled heat exchanger that cools the system refrigerant. Refrigeration compressor devices and heat exchangers are known in the art, and can be used for regulating temperature in the system 1.

The temperature regulation system can further include a pump 37 to circulate, for example, the cooled medium 35 through pass-through lines 32 and throughout the cooling/heating device 70 or chamber 10. In some embodiments, the lines 32 can be opaque to microwave irradiation. In some embodiments, the lines 32 can be largely or partially translucent to microwave irradiation. In some embodiments, the temperature regulation system 30 and/or associated lines/tubing 32 can be enclosed, for example, in a vacuum, in a dry gas environment, or with insulation. Such enclosure can be useful for controlling condensation.

The temperature regulation system can be manual or programmable, or both manual and programmable. In some automated systems, a temperature sensor 95 can be used for feedback control of temperature. In some embodiments, the cooling system can include a controller 90 that controls the flow rate of, for example, liquid or gaseous nitrogen through the lines and cooling/heating device. A temperature sensor 95 can provide temperature data back to the controller 90. The controller 90 can be configured to respond to the temperature sensor 95 feedback from the holder or a sample probe to maintain a suitable temperature at the holder. Although the controller 90 and temperature regulation system 30 are shown as separate devices in FIG. 1A, the skilled artisan will appreciate that the temperature sensor can directly provide data to a regulator within the temperature regulation system to control the temperature of the cooling/heating device. In some embodiments, the temperature sensor can be largely microwave resistant.

The operating range of the system 1 is not meant to be limited to any particular temperature, and generally is based on the phase properties of substances in the sample mixture. For example, for samples suspended in acetone, the range can be from about −200, to about 20° C. (e.g., at least, greater than, less than, or equal to about −200, −150, −100° C., −90° C., −80° C., −70° C., −60° C., −50° C., −40° C., −30° C., −20° C., −10° C., 0° C., 10° C., or 20° C.). In some embodiments in which the system is used for polymerizing embedding resin that has been infiltrated into samples, the range can be from about from about −200, to about 20° C. (e.g., at least, greater than, less than, or equal to about −200, −150, −100° C., −90° C., −80° C., −70° C., −60° C., −50° C., −40° C., −30° C., −20° C., −10° C., 0° C., 10° C., or 20° C.) for low temperature acrylic resins to about 60-100° C. (e.g., 60° C., 70° C., 80° C., 90° C., or 100° C.) for epoxy resins.

In some embodiments, one or more sample holders 75 can be used to hold one or more samples during processing. One embodiment of a sample holder 75 is shown in FIG. 1B. Preferably, the sample holder 75 includes one or more openings, such as wells or slots, and the openings 76 are configured to receive one or more samples 40. In some embodiments, multiple samples can be added to the sample holder. As shown in FIG. 1A, the sample holder(s) 75 carrying one or more samples 40 can be placed in a recess 78 in the cooling/heating device 70 and effectively seal the sample(s) 40 in the cooling/heating device 70.

In some embodiments, one or more reagents can be circulated in the system 1 to contact one or more samples. For example, reagents such as chemical compositions can be disposed in the sample holder for substitution and/or dehydration of samples. In some embodiments, the microwave processors can include conventional chambers utilizing manual exchange of reagents. In other embodiments the microwave processors can include automated systems involving mechanized reagent exchange. Such automated reagent exchange systems are known in the art and can be adapted for use with the microwave-assisted cryo-sample processing system. In some embodiments, a reagent exchange system 27 can include or be connected to a controller 90 that controls the flow rate of, for example, a chemical substance through the lines 28 and sample holder. The controller 90 can be configured to regulate the exchange of reagents as needed. In some embodiments, the reagent exchange system can be partially automated and can include options for manually overriding any automated features.

The reagents used in conjunction with the system 1 are not meant to be limited to any particular reagents, and will vary depending on the sample being processed, the particular application, etc. For example, for the preparation of biological samples for microscopy, the reagents can include chemical compositions such as dehydration reagents, fixatives, and resins. In some embodiments, the chemical composition can include an organic solvent such as, for example without limitation, acetone, methanol or ethanol. The chemical composition can further include one or more compounds for fixing a sample, such as, for example without limitation, O_{S}O₄ (osmium tetroxide), uranyl acetate, tannic acid, glutaraldehyde, or paraformaldehyde. In some embodiments, the chemical composition can include one or more resins at varying concentrations. Generally, the reagent used in contact with the sample can vary depending on the stage of sample processing. In some embodiments, the reagent is pre-cooled before contacting the samples.

In some embodiments, the system 1 is configured such that a sample can be substantially impregnated by a chemical composition in less than about 4-6 hours. In some embodiments, the system 1 is configured such that a sample can be substantially impregnated by a chemical composition in less than about two hours. In some embodiments, the system 1 is configured such that a sample can be substantially impregnated by a chemical composition in less than about 1.5, 1 or 0.5 hours. In some embodiments, the system 1 is configured such that a sample can be substantially impregnated by a chemical composition in less than, greater than or equal to about thirty, twenty-five, twenty, fifteen, ten, five, four, three, two or one minute(s). In some embodiments, impregnation with reagent is carried out under cryo conditions. In other embodiments, impregnation with reagent is carried out at ambient temperature.

In some embodiments, the system 1 for microwave-assisted cryo-sample processing can be controlled by user defined processing parameters via the controller 90. Thus, the system can be partially or fully automated for microwave-assisted cryo-sample processing, including change of reagents and exposure to microwave radiation with user defined processing parameters. In some embodiments, the system can be programmable for desired time periods and temperature settings for optimal processing. The system can also be programmable for pressure settings, moisture control settings, vapor evacuation, sample loading, etc. In some embodiments, the controller 90 can include a program storage function. In other embodiments, the system 1 can be controlled manually by user input at each stage in processing. In some embodiments, the system 1 can include safeguards for accommodating problems which may arise during processing such as, for example, exceeding desired temperature ranges, refrigerant pressure or leakage problems, power failures, etc.

In some embodiments, the system 1 can include an enclosure for the samples and holder to control condensation, isolate vapors and/or evacuate vapors. For example, the chamber 10 can include a venting system 96 that can be used to remove toxic vapors from toxic samples. In some embodiments, the venting system 96 can be attached to the sample holder. In other embodiments, the venting system can be located within the chamber 10.

In some embodiments, the chamber 10 can include removable vacuum chambers. In other embodiments, a vacuum attachment 97 can be connected to the chamber 10. The vacuum attachment can be directly attached to a sample holder. The vacuum chambers and/or vacuum attachment can regulate sample pressure. In other embodiments, the chamber 10 can include a vacuum chamber of sufficient size to accommodate the sample holder(s), and provide a sealed pass-through for coolant lines. In some embodiments, the chamber 10 can include a pressurized air system. In some embodiments, a pressurized air system can be connected to the chamber 10. In some embodiments, a system for pressurizing air or other gasses can be connected to the system 1. A pressurized air system can be directly attached to a sample holder 75. The pressurized air system can regulate sample pressure.

In some embodiments, the system 1 can include a dry-gas purge system 98 using a substance such as, for example, nitrogen gas to provide a reduced moisture environment within the oven chamber 10. Although the optional venting, vacuum and purge systems shown in FIG. 1A are depicted as external to the chamber 10, in alternative embodiments they can be placed within the microwave chamber 10. In some embodiments, devices placed within the microwave chamber for controlling condensation, vapors, etc. can be constructed from materials that are largely translucent to microwaves, or opaque to microwaves. The optional vacuum chamber/attachment, dry-gas purge system and venting systems can be connected to a controller 90 for automatic control of the various systems.

In some embodiments, a computer program is included (or can be provided separately) that controls the various parameters for microwave-assisted cryo-sample processing. In some embodiments, the program accepts user input for each factor, for example, temperature, microwave power, length of microwave irradiation, moisture level, pressure level, and reagent exposure. In this manner, cryosample processing can be automated. In some embodiments, the program performs any of the methods described herein.

An exemplary device for microwave-assisted cryo-sample processing includes a chamber that receives microwave irradiation, and a cooling/heating device disposed in the chamber to cool a sample during microwave processing of the sample. The cooling/heating device includes recesses for placement of one or more sample holders. The cooling/heating device regulates the temperature of the sample holder and samples inside the sample holder. The temperature of a sample holder is regulated by a temperature regulation system, which delivers a temperature regulating medium to the cooling/heating device in the microwave chamber. A cooling/heating medium is passed through the walls of the microwave chamber to the cooling/heating device via lines or tubing.

The sample holder includes a temperature sensor that can be used for feedback control of temperature. The device further includes an automated reagent exchange system connected to the sample holder for dispensing and removing a variety of chemical compositions into the chamber. The system also includes a venting system that can be used to remove toxic vapors from toxic samples. A vacuum attachment is connected to the sample holder. The vacuum attachment can be used to regulate sample pressure. The system also includes a dry-gas purge system using a substance such as, for example, nitrogen gas to provide a reduced moisture environment within the oven chamber.

The device can be controlled by user defined processing parameters via a controller, which can be attached to, for example, the temperature sensor, the temperature regulation system, and the reagent exchange system. The controller can also be attached to a microwave source. Thus, the system can be partially or fully automated for microwave-assisted cryo-sample processing, including change of reagents and exposure to microwave radiation with user defined processing parameters. The system can be programmed for desired time periods, temperature settings, reagent changes, pressure settings, vapor evacuation and moisture control for optimal processing.

### Sample Holders

In some embodiments, holders for holding frozen samples during microwave-assisted cryo-sample processing are provided. The holders separate and orient the samples such that samples are reproducibly uniformly fixed during microwave-assisted cryo-sample processing. The holders are generally sized to fit within microwave oven chambers.

The holders can be designed to accommodate various sizes of sample containers such as, for example without limitation, microfuge tubes, cryovials, Beem® capsules, freezer hats, and other containers. In some embodiments, the holders can include individual wells to accommodate sample containers. In other embodiments, the holders can include slots to accommodate sample containers. The dimensions of the sample holder, its slots and/or wells are not limited to any particular shapes or sizes, and are generally sized to accommodate the size and shape of the samples to be treated. The size, shape and configuration of the sample holder can vary depending on, for example, the nature of the samples, the microwave chamber size, the temperature regulation system, etc. In some embodiments, a well has a diameter of, for example without limitation, less than, greater than or equal to about 100, 99, 98 97, 96, 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1 mm. In some embodiments, a well can have a depth of, for example without limitation, less than, greater than or equal to about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 mm. Exemplary dimensions of the sample holder 310 include, without limitation, lengths about less than, greater than or equal to about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 and widths less than, greater than or equal to about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100.

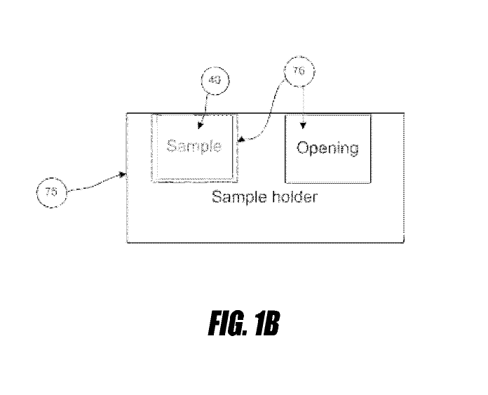

In some embodiments, the sample holders can be placed into another container, such as, for example, a cryovial. The cryovial can then be placed into a well or slot of a cooling/heating device. In other embodiments, the sample holders can be directly placed on or in a cooling/heating device. In some embodiments, multiple sample holders can be placed in recesses of a cooling/heating device. With reference to FIG. 2, for example, a sample holder 200 for microwave-assisted cryo-sample processing is illustrated. The sample holder provides for insertion of flat specimen high-pressure metallic freezing carriers 210 (“hats”) such that the frozen samples can be separated and oriented equivalently in cryovials used for microwave-assisted cryo-sample processing. The sample holder can be placed into, for example, a cryovial, and the cryovial can be placed into a cooling/heating device for keeping samples frozen during microwave-assisted cryo-sample processing. The sample holder 200 can accommodate, for example, cryo-sample hats 210 such that the samples in the wells 230 of the hats are oriented toward the microwave radiation source. In some embodiments, the sample holders 200 allow placement and simultaneous processing of multiple sample hats 210. For example, sample hats can be placed within wells or within slots 220 that can hold the hats firmly, yet allow easy sample positioning and removal from the holder. Preferably, the wells and/or slots are sized to accommodate the hats. For example without limitation, for hats about 2.8 mm in diameter and about 1-2 mm tall, the wells or slots can be about 3 mm in diameter and about 2 mm deep.

One way such cryo-sample holders were fabricated is as follows. Polytetrafluoroethylene (PTFE) blocks were obtained from Ted Pella, Inc. (Cat. #36129) and trimmed with a knife to 8 mm×5 mm×20 mm to fit within standard 2 ml cryo-vials (Thermo Fisher Scientific, Rochester, N.Y.). Small slots (FIG. 2, 200) were tooled into the long edges of the blocks to accommodate high pressure freezing sample hats (FIG. 2, 210). The slots were created using a Model 2 rotary tool, and number 110 bit (Dremel, Inc., Racine, Wis.). Sample hats were placed in these slots using tweezers under liquid nitrogen, and were retained in place by the inner walls of the cryo-vials. Variable fixation among pellets processed in the same cryovial was eliminated using these holders.

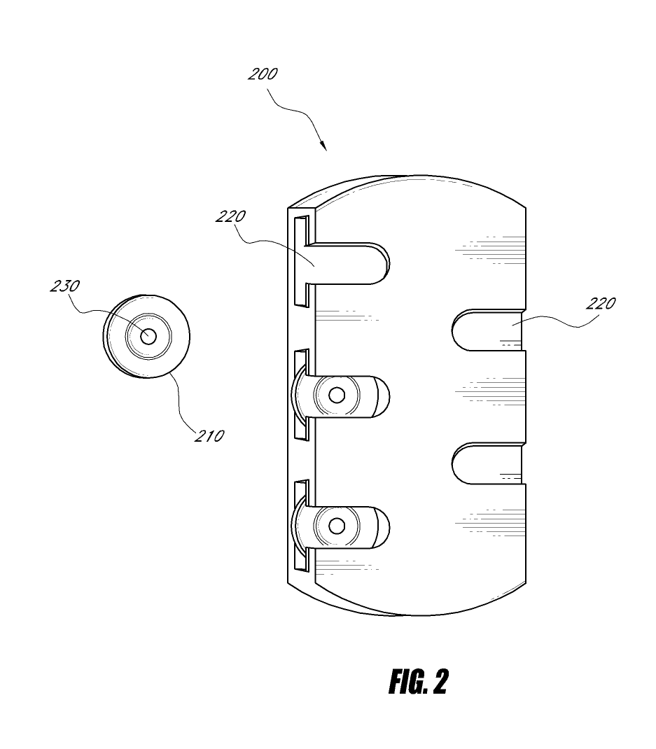

In other embodiments, a cooling/heating device such as, for example, a block or plate, can function as a sample holder. An exemplary sample holder includes a block having a plurality of wells to accommodate samples in a sample container such as, for example, a microfuge tube, a cryovial, a Beem® capsule, a freezer hat, or other container. The holder separates and orients samples such that samples are reproducibly uniformly fixed during microwave-assisted cryo-sample processing. The sample holder can also be configured to function as a cooling/heating device. With reference to FIG. 3, for example, a sample holder 310 for microwave-assisted cryo-sample processing of samples in microfuge tubes is illustrated. The sample holder includes a plurality of wells 330 for insertion of samples. The wells 330 are sized and shaped to accommodate varying sizes and shapes of samples containers (for example, microfuge tubes or cryovials). The sample holder also includes a well 360 which can be used to accommodate, for example, a temperature sensor. Exemplary dimensions of the sample holder 310 include lengths ≧6 cm and widths ≧8 cm, however, these dimensions are not meant to be limiting.

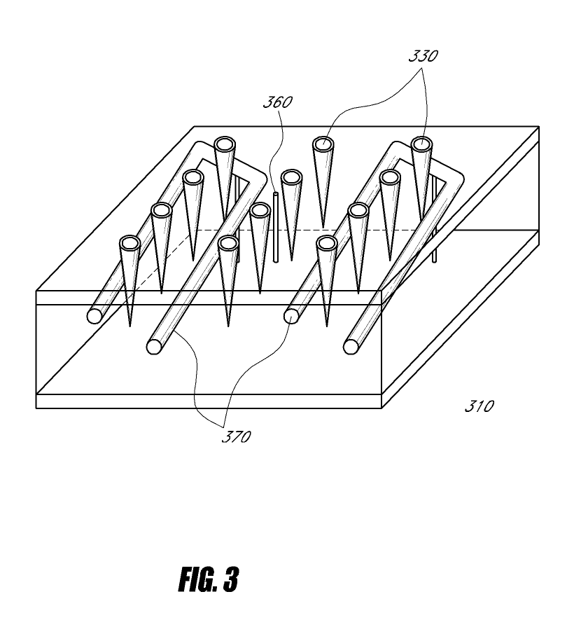

The sample holder can also be configured to function as a cooling/heating device that is adapted for convenient reagent exchange. With reference to FIG. 4, for example, a sample holder 400 for microwave-assisted cryo-sample processing is illustrated. The sample holder includes a block 410 having a plurality of wells 430 for insertion of samples. In the depicted embodiment, a well 430 can have a diameter of, for example, about 3 mm and is about 2 mm deep, and can accommodate cryosample hats. When placed into the wells, the samples will be orientated toward the microwave source, for example, upward. The wells are located within channels 440 on the top portion of the block for a reagent to allow permeation of the reagent through samples in the wells 430. For example, a reagent can be added to the channels 440 and allowed to permeate the samples in the wells 430. The sample holder 510 can have a recess 450 that can be used for reagent exchange. For example, a reagent can be delivered up through the recess 450 and flow into the wells 430. The wells 430 can have a drainage system at the bottom to allow reagent to drain out.

The sample holder can further include internal tubing as shown in FIG. 3 for a cryogenic substance to allow circulation of the substance through the sample holder for even and consistent temperature control of the sample containers. The sample holder can further include internal tubing or channels 370 for a cryogenic substance to allow circulation of the substance through the sample holder for even and consistent temperature control of the sample containers.

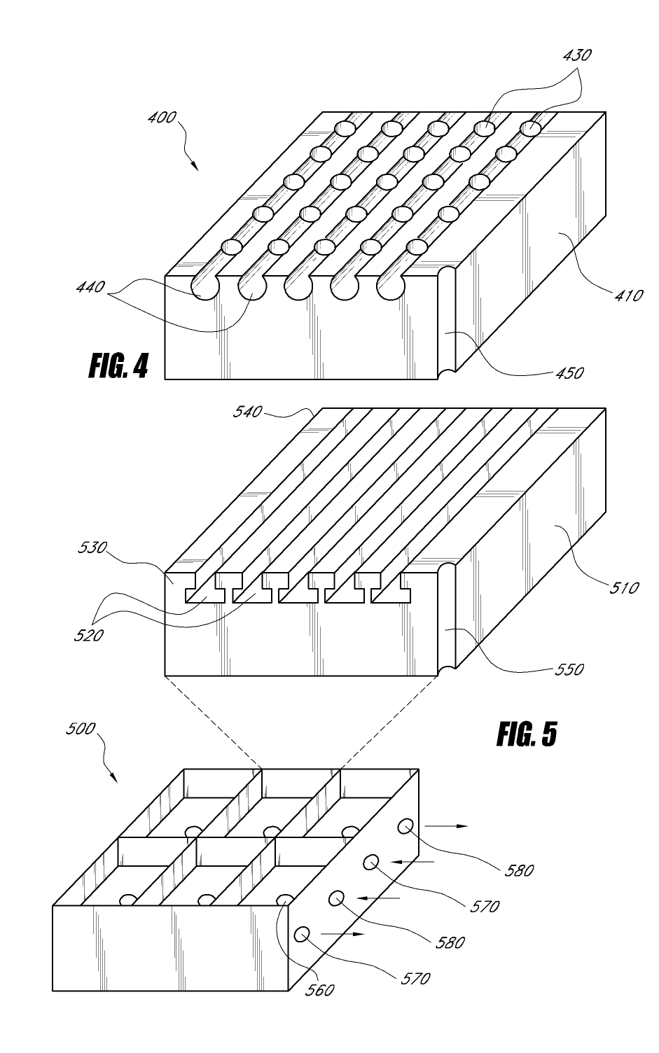

With reference to FIG. 5, for example, a cooling/heating device 500 and sample holder 510 for microwave-assisted cryo-sample processing is illustrated. The sample holder 510 includes slots 520 for insertion of samples. The sample holder 510 has a width of, for example, about 2.5 cm and a height of about 6 mm. The slots 520 extend from one end 530 to an opposite end 540 of the sample holder 510. The slot 520 has a diameter of, for example, about 3 mm and is about 2 mm deep, and can accommodate cryosample hats. Sample hats can be placed within the slots 520 that can hold the hats firmly, yet allow easy sample positioning and removal from the holder. For example, the hats can be inserted by sliding into a slot 520 from a front end 530. Once placed into the slots, the samples will be orientated toward the microwave source (upward). The cooling/heating device 500 can accommodate a plurality of sample holders 510 for simultaneous processing. The block 500 is, for example, approximately 8 cm wide and about 6 mm in height. Each sample holder 510 can have a recess 550 that can be used for reagent exchange. The cooling/heating device 500 can have a channel 560 to facilitate reagent exchange. The cooling/heating device includes inlets 570 and outlets 580 to allow circulation of a substance. The inlets and outlets can be used to circulate, for example, a cooling/heating medium through the block for even and consistent temperature control of the sample containers. For temperature control, the inlets and outlets can be connected to internal tubing. Inlets and outlets can also be used to circulate a reagent for processing of samples. For sample processing, the inlets and outlets can be connected to reagent channels 560.

The inlet and outlet for a cryogenic substance can accommodate couplers to which tubing is easily and reversibly attached. The tubing can be used to conduct a cryogenic substance from, for example, an external temperature regulation system through the sample holder, and back to the temperature regulation system. The tubing can also be used to conduct a reagent from, for example, an external reagent exchange system through the sample holder, and back to the reagent exchange system.

The sample holder can include a well for a temperature sensor that can be used for feedback control of temperature. The holder can be made from Teflon® or similar material and is thus chemically resistant and translucent to microwave irradiation. The sample holder can include internal tubing or internal channels for a cryogenic substance to allow circulation of the substance through the block for even and consistent temperature control of the sample containers. Inlets and outlets on the sample holder can accommodate couplers to which tubing is easily and reversibly attached. The tubing can be used to conduct the cryogenic substance from, for example, an external temperature regulation system through the sample holder, and back to the temperature regulation system. In other embodiments, the sample holder can have an internal temperature regulation system. The sample holder can include recesses for a reagent to allow the reagent to contact a sample. Inlets and outlets on the sample holder can accommodate couplers to which tubing is easily and reversibly attached. The tubing can be used to conduct the reagent from, for example, an external reservoir through to the sample in the sample holder, and back to the external reservoir. Thus, the cooling/heating device itself can include one or more openings such as, for example, wells, slots, etc., for insertion of samples.

In some embodiments, the holders can be made from material(s) that are chemically resistant. In some embodiments, the holder material can be, for example without limitation, translucent, or nearly translucent, to microwave irradiation. In some embodiments, the holders can be made from Teflon® or similar material. In some embodiments, the holders can withstand temperatures ranging from about 100 to about −200° C. or below. In some embodiments, the holders can accommodate gas or liquid cooling/heating medium. In some embodiments, the holders can accommodate, for example without limitation, gas, liquid, or thermoelectric cooling/heating media.

In some embodiments, the holders can include an opening to accommodate a temperature sensor. Alternatively, a temperature sensor can be constructed to mimic a sample container placed within the holder. For example, the temperature sensor can be a microfuge tube containing the probe and a defined volume of substance equivalent to that being processed, for example without limitation, 0.5 ml of acetone. The temperature sensor can be connected to, for example, a controller or regulator in the temperature regulation system and provide information for feedback control of sample holder temperature.

In some embodiments, the holders can include internal tubing or internal channels for a cryogenic substance to allow circulation of the substance through the holder for even and consistent temperature control of the sample containers. Inlet(s) and outlet(s) on the sample holder can accommodate couplers to which tubing is easily and reversibly attached. The tubing can be used to conduct the cryogenic substance from, for example, an external temperature regulation system through the sample holder, and back to the temperature regulation system.

In some embodiments, the holders can include recesses, tubing, and/or channels for a reagent to allow the reagent to contact a sample. In some embodiments, wells of a holder can include an opening at the bottom for drainage of reagent. In some embodiments, the sample holder includes a mesh, porous, or wick-lick bottom that can allow reagent permeation. Wells can be located in channels for efficient application of reagent. In some embodiments, reagent can be added manually to the samples. In some embodiments, the sample holder can have, for example, a recess to facilitate application of reagent. The recess can be connected to a reagent exchange system. Inlet(s) and outlet(s) on the sample holder can accommodate couplers to which tubing is easily and reversibly attached. The tubing can be used to conduct the reagent from, for example, an external reservoir through to the sample in the sample holder, and back to the external reservoir.

In some embodiments, a pipette or similar device can be inserted into the bottom of a well in a sample holder to remove and add reagents manually. In some embodiments, a robotic transfer device can be used for reagent exchange. Reagent exchange can also be accomplished manually or automatically, for example, by draining the well through the block, then refilling with the next reagent via channels in the block. In some embodiments, the temperature of the replacement reagent can be cooled or heated before introducing it to the samples.

The holders themselves, and the holder inlets, outlets, and tubing can be manufactured from material such as, for example without limitation, Teflon® (PFTE) or other microwave-translucent material, ceramics, glass, plastics, fabrics and metals. Preferably, the holder is made from one or more materials that are translucent, or nearly translucent, to microwave irradiation. In some embodiments, the holders are structurally and chemically durable. In some embodiments, the holder is easily molded or tooled and/or capable of withstanding temperatures ranging from about 100 to about −200° C. or below. In some embodiments, the sample holders can include a gas-filled or evacuated enclosure to minimize or eliminate condensation.

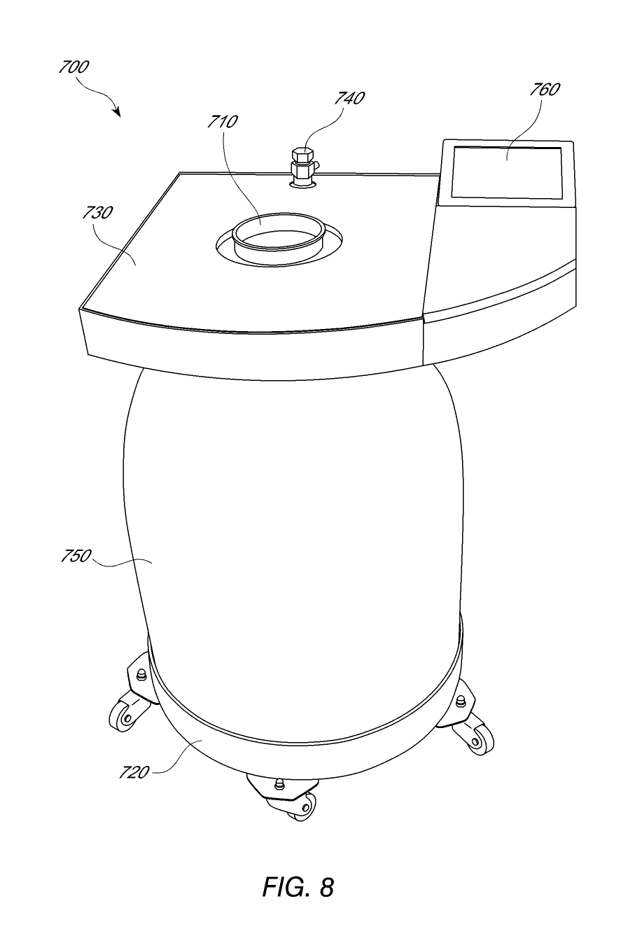

With reference to FIG. 8, for example, a cooling/heating device 700 and cryo-chamber 710 for microwave-assisted cryo-sample processing is illustrated. The cooling/heating device 700 has a stage 730, which may be approximately 21 inches wide, 16¾ inches to (at a widest point) 18¼ inches deep, and 2⅓ inches thick. A liquid nitrogen port 740 can be located on the stage and is connectable to a liquid nitrogen container 750, which may be placed in a wheeled stand 720. A viewing screen 760 can be located on the stage and can be configured to have dimensions, such as, 6½ inches width and 3¼ inches height. The viewing screen 760 can extend beyond the stage 730 by approximately 1¾ inches in some embodiments.

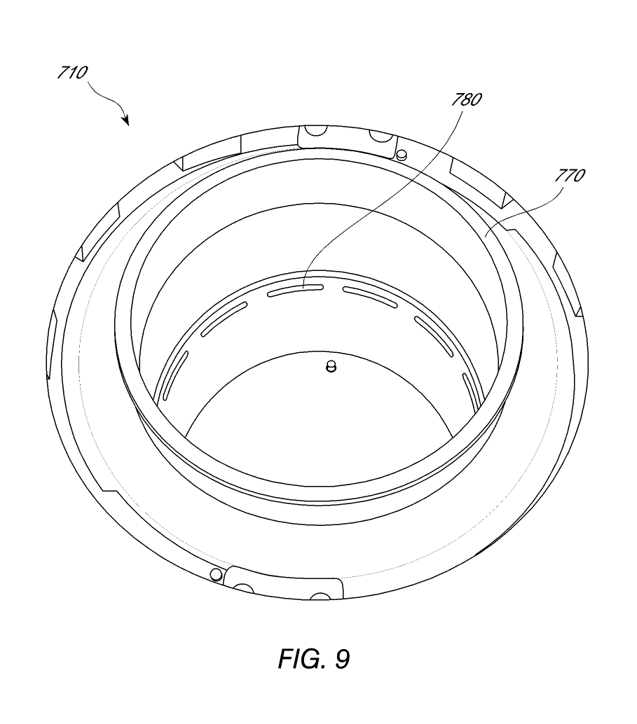

With reference to FIG. 9, for example, the cryo chamber 710 can have an interior diameter of, for example, about 3⅞ inches, a rim 770 diameter of about 4¼ inches, and a total recess diameter of about 5¼ inches. The cryo-chamber 710 may also have slots 780, which slots 520 extend along the entire interior circumference of the cryo-chamber 710. The cryo-chamber 710 may also have a glass cover.

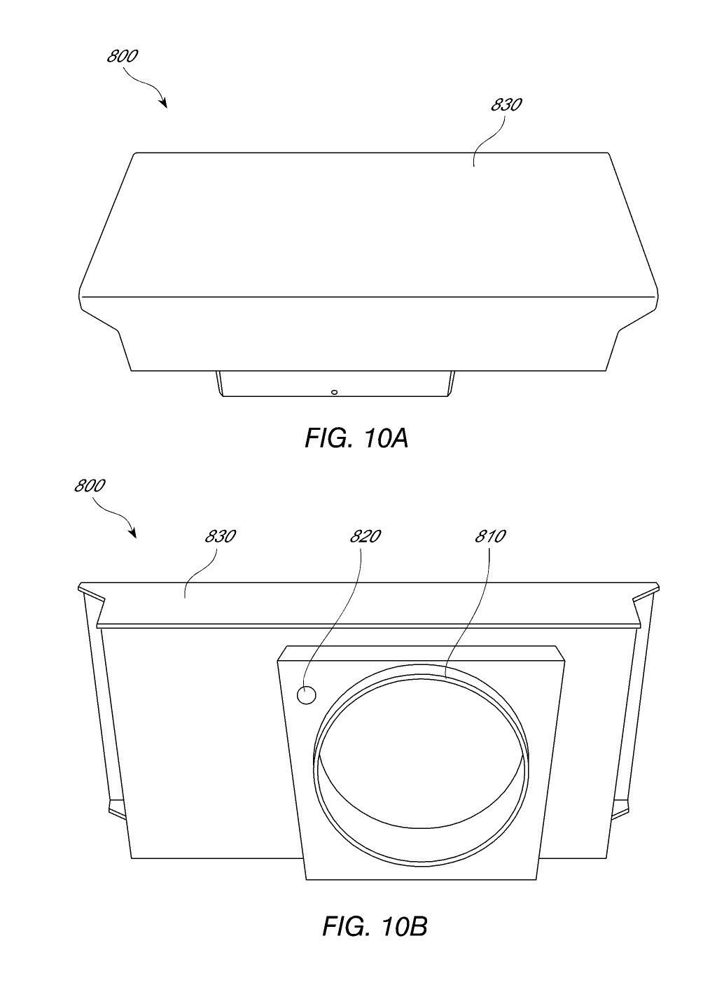

With reference to FIG. 10, for example, a microwave irradiation unit 800 that is configured to attach to the cryo-chamber 710 is illustrated. Microwave irradiation unit 800 can have a housing 830, which encases the microwave emitting device, and cryo-chamber interface 810, which connects to the cryo-chamber 710. The cryo-chamber 710 is made from material that won't interfere with or be damaged by the microwave radiation (e.g., polypropylene or glass). Preferably, the microwave irradiation unit 800 has a safety interlock sensor 820, which is configured to allow for the transfer of power to the microwave irradiation unit 800 when the sensor 820 is in an engaged position. That is, in some embodiments, the safety interlock sensor is configured to regulate the operation of the microwave irradiation unit 800 only when the microwave irradiation unit 800 is engaged with the cryo-chamber 710. The microwave irradiation unit 800 also has, preferably, an on and off switch, a breaker control, and connections to a power source and automated controller (not shown). Also, in some embodiments, additional ports and conduits connect to the cryo-chamber 710 and these ports and conduits are configured to allow automated reagent exchange within the chamber, without having to remove the microwave irradiation unit 800.

### Methods

Microwave-assisted cryo-sample processing can be used for a variety of different applications. For example without limitation, microwave-assisted cryo-sample processing can be used for freeze substitution (i.e., MWFS) of all types of frozen samples applicable to study by cryo-electron microscopy including but not limited to any biological material and non-biological aqueous materials (e.g., bacterial cell samples, human cell samples, mammalian cell samples, viruses, animal, preferably, mammalian tissues, such as human tissues, hydrogels, fungi, protozoans, prions, subcellular organelles, bioproducts, or biomolecular complexes). Accordingly, aspects of the invention include methods for microwave-assisted cryo-sample processing.

In some embodiments, the dissolution of ice in a frozen specimen by an organic solvent during microwave-assisted cryo-sample processing can be carried out at temperatures below which secondary ice crystals may grow, i.e., below about −70° C. Preferably, the organic solvent is cooled prior to contact with the samples (e.g., a bacterial cell or human cell). The temperature of steps during microwave-assisted cryo-sample processing can range from about −10° C. to about −200° C. or below. In some embodiments, a microwave-assisted cryo-sample processing step can be carried out at less than, greater than or equal to about −200° C. and about +20° C. (e.g., at least, greater than, less than, or equal to about −200, −150, −100° C., −90° C., −80° C., −70° C., −60° C., −50° C., −40° C., −30° C., −20° C., −10° C., 0° C., 10° C., or 20° C.). The temperature can be varied to optimize incubation temperature with the reactivity of fixative reagents. The temperature range of processing steps can depend on the solvent used. For example, ethanol has a freezing point of −114° C. and acetone has a freezing point of −94.7° C. In some embodiments, epoxy resin polymerization can be conducted at, for example, about 60-70° C. In some embodiments, acrylic resin polymerization can be conducted at, for example, about 0-20° C. under UV light, or at about 60° C. or higher. In other embodiments, polymerization can be conducted at, for example, about 100° C. Paraffin is typically infiltrated at elevated temperature and then cooled to harden.

In some embodiments, once substitution with solvent is complete, samples can be warmed up without recrystallization, as water is now absent from the sample. For example, samples intended for immunocytochemistry can be infiltrated with resin and polymerized can also include steps at ambient temperature. In other embodiments, infiltration with resin can be carried out at low temperature to reduce any damaging effects that ambient-temperature organic solvents and heat polymerization may have on some epitopes.

The power level of the radiation used to irradiate the frozen samples can vary, and can typically range, for example without limitation, from about 0 to 750 W and in some embodiments, 0-1500 W or 0-2000 W. In some embodiments, the microwave irradiation can be constant. In other embodiments, the microwave irradiation can be pulsed. In some embodiments, a power level of about 65, 70, 75, 80, 85, or 90 W is used. The power level of the radiation used to irradiate the substituted samples during infiltration of a subsequent substance, such as, for example, resin, can vary. In some embodiments, a power level of about 150, 200, 225, 250, 275, or 300 W is used. The skilled artisan will appreciate that a variety of power levels can be used, constant or pulsed, and the wattage, time, and heat load control can be varied to optimize processing.

The amount of continuous irradiation time during substitution can vary from about 30 sec to about 5 minutes. In some embodiments, a sample can be continuously irradiated for less than, greater than or equal to about 0, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, 10 or more minutes. For microwave-assisted cryo-sample processing, a radiation series is performed with frozen samples under cryo conditions for substitution with solvent. In some embodiments, the radiation series can include a first period of irradiation, a period of rest during which there is no irradiation, and a second period of irradiation.

In some embodiments, the radiation can be constant. In other embodiments, the radiation can be pulsed. For example, a sample can be continuously irradiated in multiples of 3 minute cycles. However, the irradiation cycle used will varying depending on the sample, solvent, process, etc. and is not meant to be limited to any particular parameters.

In some embodiments, the radiation series can include, for example: about 30 sec to about 5 min microwave irradiation on, about 30 sec to about 5 min microwave irradiation off, about 30 sec to about 5 min on. In some embodiments, the radiation series includes is as follows: about 2 min microwave irradiation on, about 2 min microwave irradiation off, about 2 min on. The radiation series can be performed one, two, three, four, five, six, seven, eight, nine, ten or more times. In some embodiments, the radiation series is performed at least two times. In some embodiments, the radiation series is performed less than, greater than or equal to about two, three, four, five, six, seven, eight, nine, ten eleven, or twelve times. In other embodiments, the length of irradiation can vary between series. For example, a first series can include: about 2 min microwave irradiation on, about 2 min microwave irradiation off, about 2 min on, and a second series can include: about 1 min microwave irradiation on, about 2 min microwave irradiation off, about 1 min on.

In some embodiments, a frozen sample can be substituted with a solvent using the following radiation series at a power level of about 80 W: about 2 min microwave irradiation on (on), about 2 min microwave irradiation off, about 2 min on. The series can be repeated at least one time.

During microwave irradiation, the sample(s) can be contacted with one or more reagents, such as chemical composition(s), for processing. For at least a portion of the process, the processing occurs under cryo-conditions. For example, substitution and dehydration can take place under cryo-conditions, and subsequent resin exchange can take place at ambient or reduced temperature. For example, acrylic resins are often infiltrated at about 4° C. or lower. The chemical composition can be disposed within the chamber and/or sample holder and contact the sample. In some embodiments, the chemical composition can include an organic solvent such as, for example without limitation, acetone, methanol or ethanol. The chemical composition can further include one or more compounds for fixing a sample, such as, for example without limitation, O_{S}O₄ (osmium tetroxide), uranyl acetate, tannic acid, glutaraldehyde, or paraformaldehyde. In some embodiments, the chemical composition can include O_{S}O₄ (osmium tetroxide) in acetone. In some embodiments, the chemical composition can include a resin. As discussed above, the reagents used during processing will vary depending on the particular sample and application.

In some embodiments, after an irradiation cycle, the reagent in contact with the sample can be exchanged for a fresh batch of the same reagent, or different reagent. For example, an initial reagent can include a fixative, and later reagents can include a dehydrating reagent without fixative. Still later reagents can include, for example, increasing concentrations of resin. Reagents for embedding sample include, without limitation, epoxy resins, acrylic resins, paraffin and other substances.

The irradiation cycles and total length of microwave irradiation time can vary and depends on the chemical composition used, temperatures, etc. In some embodiments, the sample can be impregnated by the chemical composition in less than about two hours. In some embodiments, sample is substantially impregnated by the chemical composition in less than about twenty minutes. Specific reagents and irradiation cycle conditions are provided below in the Examples.

### EXAMPLES

Embodiments of the present teachings can be further understood in light of the following examples, which should not be construed as limiting the scope of the present teachings in any way.

### Example 1

### Device for Microwave-Assisted Cryo Processing

This Example illustrates one embodiment of a device for microwave-assisted cryo-sample processing. An exemplary device for microwave-assisted cryo-sample processing includes a chamber that receives microwave irradiation, and a cooling/heating device disposed in the chamber to cool a sample during microwave processing of the sample. The cooling/heating device includes recesses for placement of one or more sample holders. The cooling/heating device regulates the temperature of the sample holder and samples inside the sample holder.

The temperature of a sample holder is regulated by a temperature regulation system, which delivers a temperature regulating medium to the cooling/heating device in the microwave chamber. A cooling/heating medium is passed through the walls of the microwave chamber to the cooling/heating device via lines or tubing.

The sample holder includes a temperature sensor that can be used for feedback control of temperature. The device further includes an automated reagent exchange system connected to the sample holder for dispensing and removing a variety of chemical compositions into the chamber. The system also includes a venting system that can be used to remove toxic vapors from toxic samples. A vacuum attachment is connected to the sample holder. The vacuum attachment can be used to regulate sample pressure. The system also includes a dry-gas purge system using a substance such as, for example, nitrogen gas to provide a reduced moisture environment within the oven chamber.

The device can be controlled by user defined processing parameters via a controller, which can be attached to, for example, the temperature sensor, the temperature regulation system, and the reagent exchange system. The controller can also be attached to a microwave source. Thus, the system can be partially or fully automated for microwave-assisted cryo-sample processing, including change of reagents and exposure to microwave radiation with user defined processing parameters. The system can be programmed for desired time periods, temperature settings, reagent changes, pressure settings, vapor evacuation and moisture control for optimal processing.

### Example 2

### Sample Holder for Microwave-Assisted Cryo Processing

This Example illustrates one embodiment of a sample holder for microwave-assisted cryo-sample processing. With reference to FIG. 2, an exemplary sample holder 200 for microwave-assisted cryo-sample processing is illustrated. The sample holder provides for insertion of flat specimen high-pressure metallic freezing carriers 210 (“hats”) such that the frozen samples can be separated and oriented equivalently in cryovials used for microwave-assisted cryo-sample processing. The sample holder can be placed into, for example, a cryovial, and the cryovial can be placed into a cooling/heating device for keeping samples frozen during microwave-assisted cryo-sample processing. The sample holder 200 can accommodate, for example, cryo sample hats 210 such that the samples in the wells 230 of the hats are oriented toward the microwave radiation source. In some embodiments, the sample holders 200 allow placement and simultaneous processing of multiple sample hats 210. For example, sample hats can be placed within wells or within slots 220 that can hold the hats firmly, yet allow easy sample positioning and removal from the holder. Preferably, the wells and/or slots are sized to accommodate the hats. For example without limitation, for hats about 2.8 mm in diameter and about 1-2 mm tall, the wells or slots can be about 3 mm in diameter and about 2 mm deep.

### Example 3

### Cryo-Sample Holder Fabrication

This Example illustrates fabrication of one embodiment of a cryo-sample holder. Polytetrafluoroethylene (PTFE) blocks were obtained from Ted Pella, Inc. (Cat. #36129) and trimmed with a knife to 8 mm×5 mm×20 mm to fit within standard 2 ml cryo-vials (Thermo Fisher Scientific, Rochester, N.Y.). Small slots (FIG. 2, 200) were tooled into the long edges of the blocks to accommodate high pressure freezing sample hats (FIG. 2, 210). The slots were created using a Model 2 rotary tool, and number 110 bit (Dremel, Inc., Racine, Wis.). Sample hats were placed in these slots using tweezers under liquid nitrogen, and were retained in place by the inner walls of the cryo-vials. Using these holders eliminated variable fixation among pellets processed in the same cryovial.

### Example 4

### Sample Holder for Microwave-Assisted Cryo Processing

This Example illustrates one embodiment of a device for microwave-assisted cryo-sample processing. An exemplary sample holder includes a block having a plurality of wells to accommodate samples in a sample container such as, for example, a microfuge tube, a cryovial, a Beem® capsule, a freezer hat, or other container. The holder separates and orients samples such that samples are reproducibly uniformly fixed during microwave-assisted cryo-sample processing.

The sample holder includes a well for a temperature sensor that can be used for feedback control of temperature. The holder can be made from Teflon® or similar material and is thus chemically resistant and translucent or opaque to microwave irradiation. In some embodiments, the holder is manufactured from an Aluminum alloy that is not reactive to the microwave radiation (e.g., anodized Aluminum). The sample holder includes internal tubing or internal channels for a cryogenic substance to allow circulation of the substance through the block for even and consistent temperature control of the sample containers. Inlets and outlets on the sample holder can accommodate couplers to which tubing is easily and reversibly attached. The tubing can be used to conduct the cryogenic substance from, for example, an external temperature regulation system through the sample holder, and back to the temperature regulation system. In other embodiments, the sample holder can have an internal temperature regulation system.

The sample holder includes recesses for a reagent to allow the reagent to contact a sample. Inlets and outlets on the sample holder can accommodate couplers to which tubing is easily and reversibly attached. The tubing can be used to conduct the reagent from, for example, an external reservoir through to the sample in the sample holder, and back to the external reservoir.

### Example 5

### Sample Holder for Microwave-Assisted Cryo Processing

This Example illustrates one embodiment of a sample holder for microwave-assisted cryo-sample processing. The sample holder functions as a cooling/heating device. With reference to FIG. 3, a sample holder 310 for microwave-assisted cryo-sample processing of samples in microfuge tubes is illustrated. The sample holder includes a plurality of wells 330 for insertion of samples. The wells 330 are sized and shaped to accommodate varying sizes and shapes of samples containers (for example, microfuge tubes or cryovials). The sample holder also includes a well 360 which can be used to accommodate, for example, a temperature sensor. Exemplary dimensions of the sample holder 310 include lengths ≧6 cm and widths ≧8 cm, however, these dimensions are not meant to be limiting. The sample holder further includes internal tubing or channels 370 for a cryogenic substance to allow circulation of the substance through the sample holder for even and consistent temperature control of the sample containers.

### Example 6

### Sample Holder for Microwave-Assisted Cryo Processing

This Example illustrates one embodiment of a sample holder for microwave-assisted cryo-sample processing. The sample holder can function as a cooling/heating device, and is also adapted for convenient reagent exchange. With reference to FIG. 4, an exemplary sample holder 400 for microwave-assisted cryo-sample processing is illustrated. The sample holder includes a block 410 having a plurality of wells 430 for insertion of samples. In the depicted embodiment, a well 430 can have a diameter of, for example, about 3 mm and is about 2 mm deep, and can accommodate cryosample hats. When placed into the wells, the samples will be orientated toward the microwave source, for example, upward.

The wells are located within channels 440 on the top portion of the block for a reagent to allow permeation of the reagent through samples in the wells 430. For example, a reagent can be added to the channels 440 and allowed to permeate the samples in the wells 430. The sample holder 510 can have a recess 450 that can be used for reagent exchange. For example, a reagent can be delivered up through the recess 450 and flow into the wells 430. The wells 430 can have a drainage system at the bottom to allow reagent to drain out. The sample holder can further include internal tubing as shown in FIG. 3 for a cryogenic substance to allow circulation of the substance through the sample holder for even and consistent temperature control of the sample containers.

### Example 7

### Sample Holder for Microwave-Assisted Cryo Processing

This Example illustrates one embodiment of a sample holder for microwave-assisted cryo-sample processing. With reference to FIG. 5, an exemplary cooling/heating device 500 and sample holder 510 for microwave-assisted cryo-sample processing is illustrated. The sample holder 510 includes slots 520 for insertion of samples. The sample holder 510 has a width of, for example, about 2.5 cm and a height of about 6 mm. The slots 520 extend from one end 530 to an opposite end 540 of the sample holder 510. The slot 520 has a diameter of, for example, about 3 mm and is about 2 mm deep, and can accommodate cryosample hats. Sample hats can be placed within the slots 520 that can hold the hats firmly, yet allow easy sample positioning and removal from the holder. For example, the hats can be inserted by sliding into a slot 520 from a front end 530. One placed into the slots, the samples will be orientated toward the microwave source (upward).

The cooling/heating device 500 can accommodate a plurality of sample holders 510 for simultaneous processing. The block 500 is, for example, approximately 8 cm wide and about 6 mm in height. Each sample holder 510 can have a recess 550 that can be used for reagent exchange. The cooling/heating device 500 can have a channel 560 to facilitate reagent exchange.

The cooling/heating device includes inlets 570 and outlets 580 to allow circulation of a substance. The inlets and outlets can be used to circulate, for example, a cooling/heating medium through the block for even and consistent temperature control of the sample containers. For temperature control, the inlets and outlets can be connected to internal tubing. Inlets and outlets can also be used to circulate a reagent for processing of samples. For sample processing, the inlets and outlets can be connected to reagent channels 560.

The inlet and outlet for a cryogenic substance can accommodate couplers to which tubing is easily and reversibly attached. The tubing can be used to conduct a cryogenic substance from, for example, an external temperature regulation system through the sample holder, and back to the temperature regulation system. The tubing can also be used to conduct a reagent from, for example, an external reagent exchange system through the sample holder, and back to the reagent exchange system.

### Example 8

### Sample Processing Using a Device for Microwave-Assisted Cryo-Sample Processing

This Example illustrates processing of samples using device for microwave-assisted cryo-sample processing. In this Example, microwave processing steps are conducted in device a microwave-assisted cryo-sample processing as described in Example 1. Frozen samples are processed as shown in Table 1. Blocks and sections are subsequently prepared from the microwave processed samples for microscopy analysis. In some embodiments, the level of vacuum is maintained at about 200-550 Torr (e.g., at least, equal to, or greater than 200, 250, 300, 400, or 500 Ton).

TABLE 1

| | | | Temperature | Vacuum |
|---|---|---|---|---|
| Step | Power (W) | Time | (° C.) | (~500 torr) |
| Fixative 4-10 times | About 80 | About 1-3 min on, about 1-3 | About −85 to | − |
| (substitution) | | min off, about 1-3 min on | −65 | |
| Solvent wash | About 80 | About 30-60 sec | About −85 to | − |
| | | | −65 | |
| Solvent dehydration | About 80 | About 30-60 sec each | About 0 | − |
| twice | | | | |
| Resin 50%, 75%, | About 250 | About 2-5 min each | Ambient | + |
| 100%, 100% | | | | |

### Example 9

### Bacterial Strain

This Example illustrates growth and harvesting of a bacterial sample to be fixed using a variety of methods for microscopy analysis. In various of the Examples described herein, cultures of Bacillus subtilis, Gram positive spore-forming bacterium, were used. As with pathogenic Gram positive bacteria, B. subtilis has a thick cell wall that resists diffusion of fixatives and viscous embedding resins (Grahamm, L. L., and Beveridge, T. J., J. Bacteriol. 176: 1413-21 (1994); Matias, V. R. F., and Beveridge, T. J. Mol. Microbiol. 56: 240-51 (2006). B. subtilis provided a model organism with known cell structure and minimal biohazard potential, yet presented a significant cell wall barrier to diffusion of fixation and embedding reagents often encountered with pathogens such as staphylococci and streptococci, and many plants and fungi.

In this Example, B. subtilis strain 6051 was obtained from the American Type Culture Collection, Manassas, Va. Cultures were propagated aerobically at 37° C. in Luria broth. Mid-log phase cultures were harvested by centrifugation at 2000×g for 5 min. Pellets were washed twice in sterile Hank's buffered salt solution (HBSS) (Cambrex, Inc., Walkersville, Md.), then resuspended and centrifuged in HBSS containing 10% bovine serum albumin (Sigma-Aldrich Chemical Co., St. Louis, Mo.) (HBSS-BSA).

### Example 10

### Chemical Fixation

This Example illustrates chemical fixation of a B. subtilis sample. Pellets of Bacillis subtilis strain 6051, described in Example 1, were prepared for conventional chemical fixation by submersion in Karnovsky's fixative containing 4% glutaraldehyde, 4% paraformaldehyde, and 0.1 M sodium phosphate buffer, pH 7.2 (Electron Microscopy Sciences, Hatfield, Pa.), overnight at 4° C. The pellets were then pre-embedded in 2% NuSieve agarose (Cambrex), washed twice for 30 min each in phosphate buffer, and post-fixed for 1 hr at room temperature (22-24° C.) in 1% osmium tetroxide in phosphate buffer. The samples were then washed once in phosphate buffer, twice in water, and stained with 1% aqueous uranyl acetate. The samples were dehydrated in an ethanol series and infiltrated and embedded in Spurr's low-viscosity resin (Ted Pella, Inc., Redding, Calif.).

### Example 11

### High Pressure Freezing

This Example illustrates high pressure freezing of B. subtilis. Bacterial pellets prepared as described in Example 1 were transferred to 1.2 mm×0.4 mm flat specimen high-pressure freezing carriers (“hats”) (Leica Microsystems, Inc, Bannockburn, Ill.). The samples were immediately cryo-fixed with liquid nitrogen in a Leica model EMPact high pressure freezer. Samples frozen at less than 2000 bar or at rates less than 11,000° C. per second were discarded. Hats containing frozen bacteria were stored under liquid nitrogen until used.

### Example 12

### Standard Freeze Substitution

This Example illustrates standard freeze substitution of a sample. For standard FS, hats containing frozen bacteria prepared as described in Example 3 were transferred to cryovials containing 0.5 ml of a mixture containing frozen 1% osmium tetroxide, 0.1% uranyl acetate and acetone under liquid nitrogen. The vials were placed into the pre-cooled chamber of a model AFS automated freeze substitution instrument (Leica, Microsystems, Inc.) and slowly warmed with the following parameters: −90° C. for 12 hr, ramp to −80° C. over 2 hr, −80° C. for 12 hr, ramp to −40° C. over 20 hr, −40° C. for 39 hr. Samples were then transferred to pre-cooled 100% acetone, and ramped to 0° C. over 4 hr. Samples were then held at 0° C. for 12 hr. Samples were further dehydrated with two changes of 100% acetone, removed from the hats, infiltrated and embedded in Spurr's resin using acetone as the series solvent.

### Example 13

### Microwave-Assisted Processing

This Example illustrates microwave-assisted processing of a sample at ambient temperature. In this Example, all microwave processing steps were conducted in a model 3451 microwave processor, equipped with a ColdSpot™ load cooler and vacuum system, and with variable wattage from 80 to 750 W (Ted Pella, Inc.). Steps for chemically-fixed samples were adapted from procedures published previously by Webster, and Gibberson and collegues (Giberson, R. T., and Demaree, R. S., (Eds.) Microwave techniques and protocols, Humana Press, Springer, N.Y., (2001); Munoz et al. J. Neurosci. Methods. 137: 133-9 (2004); Webster, P. Methods. Mol. Biol. 369: 47-65. 2007). Chemically-fixed samples were processed at ambient temperature as shown in Table 2.

TABLE 2

| | Power | | Temperature | Vacuum |
|---|---|---|---|---|
| Step | (W) | Time | (° C.) | (~500 torr) |
| Karnovsky's fix | 250 | 2 min on, | Approx. 24-30 | + |
| | | 2 min off, | | |
| | | 2 min on | | |
| Same as previous | 250 | 2 min on, | Approx. 24-30 | + |
| | | 2 min off, | | |
| | | 2 min on | | |
| 2 buffer washes | 80 | 45 sec | Approx. 24-30 | + |
| Post-fix twice in 1% | 80 | 2 min on, | Approx. 24-30 | + |
| OsO₄ in buffer | | 2 min off, | | |
| | | 2 min on | | |
| Buffer wash | 80 | 45 sec | Approx. 24-30 | + |
| 2 water washes | 80 | 45 sec | Approx. 24-30 | + |
| UAc in-block stain | 80 | 2 min on, | Approx. 24-30 | + |
| twice | | 2, min off, | | |
| | | 2 min on | | |
| 2 water washes | 80 | 45 sec | Approx. 24-30 | + |
| EtOH 70, 100, 100 | 80 | 45 sec each | Approx. 24-30 | − |
| Resin 50%, 75%, | 250 | 3 min each | Approx. 24-30 | + |
| 100%, 100% | | | | |

### Example 14

### Microwave-Assisted Cryo-Sample Processing

This Example illustrates microwave-assisted cryo-sample processing. A container of crushed dry ice was tested to see if it could withstand MW irradiation while keeping water frozen. Over a 15 min period at 250 W or at 80 W, a wet ice sample remained frozen and only 7% and 8.5% of crushed dry ice was lost, respectively. A comparable sample left at room temperature without irradiations lost 8% mass. Clearly, the MW irradiation had negligible if any effect on the rate of dry ice sublimation in the system, and would not melt frozen hydrated samples encased within a crushed dry ice.

In this Example, all microwave processing steps were conducted in a model 3451 microwave processor, equipped with a ColdSpot™ load cooler and vacuum system, and with variable wattage from 80 to 750 W (Ted Pella, Inc.). Frozen samples prepared as described in Example 3 were processed as shown in Table 3. The steps at −78° C. were carried out using crushed dry ice to maintain cryo-conditions.

TABLE 3

| | Power | | | Vacuum |
|---|---|---|---|---|
| Step | (W) | Time | Temperature | (~500 torr) |
| 1% OsO4/0.1% UAc | 80 | 2 min on, | crushed dry | − |
| fixative | | 2 min off, | ice | |
| | | 2 min on | (approx. −85 | |
| | | | to −65° C.) | |
| Same as previous step | 80 | 2 min on, | crushed dry | − |
| | | 2 min off, | ice | |
| | | 2 min on | (approx. −85 | |
| | | | to −65° C.) | |
| Same as previous step | 80 | 2 min on, | crushed dry | − |
| | | 2 min off, | ice | |
| | | 2 min on | (approx. −85 | |
| | | | to −65° C.) | |
| Same as previous step | 80 | 2 min on, | crushed dry | − |
| | | 2 min off, | ice | |
| | | 2 min on | (approx. −85 | |
| | | | to −65° C.) | |
| Same as previous step | 80 | 2 min on, | crushed dry | − |
| | | 2 min off, | ice | |
| | | 2 min on | (approx. −85 | |
| | | | to −65° C.) | |
| Same as previous step | 80 | 2 min on, | crushed dry | − |
| | | 2 min off, | ice | |
| | | 2 min on | (approx. −85 | |
| | | | to −65° C.) | |
| Same as previous step | 80 | 2 min on, | crushed dry | − |
| | | 2 min off, | ice | |
| | | 2 min on | (approx. −85 | |
| | | | to −65° C.) | |
| Same as previous step | 80 | 2 min on, | crushed dry | − |
| | | 2 min off, | ice | |
| | | 2 min on | (approx. −85 | |
| | | | to −65° C.) | |
| Acetone wash | 80 | 45 sec | crushed dry | − |
| | | | ice | |
| | | | (approx. −85 | |
| | | | to −65° C.) | |
| Acetone dehydration | 80 | 45 sec each | wet ice | − |
| twice | | | (approx. | |
| | | | 0° C.) | |
| Resin 50%, 75%, | 250 | 3 min each | Ambient | + |
| 100%, 100% | | | (approx. | |
| | | | 24-30° C.) | |

### Example 15

### Preparation of Blocks and Sections

This Example illustrates preparation of blocks and sections for microscopy analysis of samples. Samples prepared as described above in Examples 1-6 were polymerized in 100% resin overnight at 65° C., sectioned with diamond knives, and examined at 80 kV with a model H7500 transmission electron microscope (Hitachi High Technologies, Pleasanton, Calif.). Images were collected with an XR-100 CCD camera system (Advanced Microscopy Techniques, Danvers, Mass.), and processes with Adobe PhotoShop® (Adobe Systems, Inc., San Jose, Calif.).

### Example 16

### Comparison of B. subtilis Sections Prepared by Various Methods

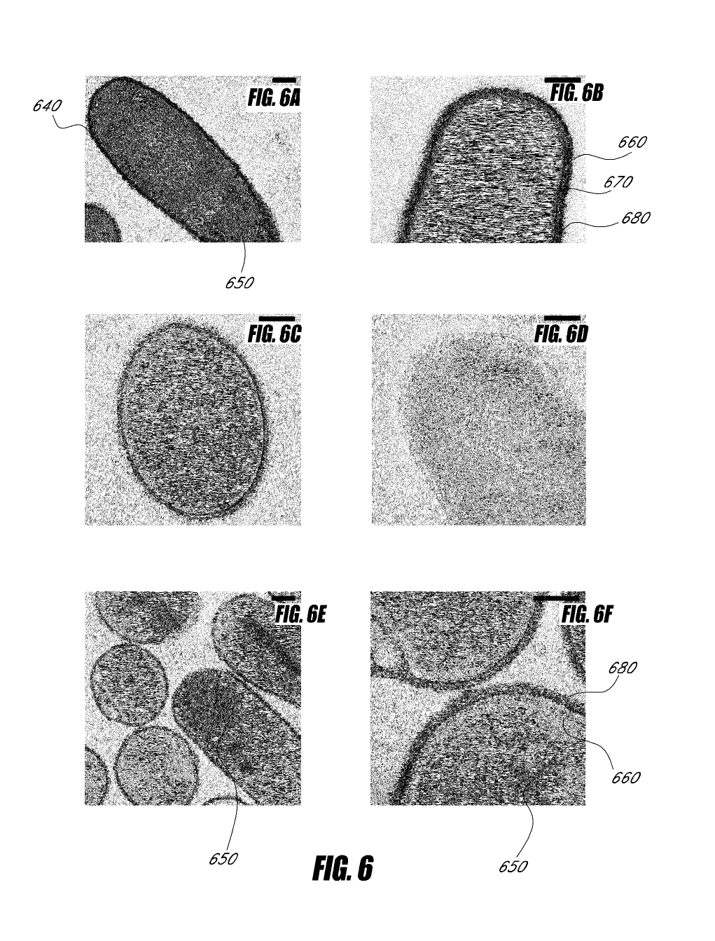

This Example illustrates results from a comparison of various methods for preparing B. subtilis sections. Images of B. subtilis sections prepared by conventional fixation, MW-assisted chemical fixation, traditional passive FS, and microwave-assisted freeze substitution (MWFS) were compared (FIG. 6). FIG. 6A-F show a comparison of samples of Bacillus subtilis that were fixed conventionally (FIG. 6A-chemical fixation at ambient temperature), fixed with standard MW assisted chemical processing (FIG. 6B-MW-assisted chemical fixation at ambient temperature), traditional passive FS (FIG. 6C), cryofixed followed rapid by FS (no microwave irradiation) (FIG. 6D), and cryofixed followed by microwave-assisted freeze substitution (MWFS) (FIGS. 6E-F). The results of MWFS (FIGS. 6E-F) showed several structural components 620 of the B. subtilis that were evident in cryofixed samples but obscured or absent in chemically fixed samples (FIGS. 6A-B). Samples prepared by MWFS were favorably comparable to standard FS. Preservation of fine structures and morphology resembled that observed with traditional freeze substitution and showed significant improvement over conventional fixation using passive diffusion or microwave processing at ambient temperature.

The figure shows that each method produced informative images of B. subtilis ultrastructure. However, there were noticeable differences. Conventional chemical fixation resulted in a relatively compact cell wall 640 and extracellular layer measuring 11-14 nm in thickness, and minimal delineation of ribosomes 650 (FIG. 6A). Samples processed by ambient MW-assisted chemical fixation exhibited a well defined plasma membrane 660 and dense layer of cell wall 670 and extra cellular material 680 measuring 18-21 nm thick (FIG. 6B). Cryo-preparations produced homogenous cytoplasm with distinguishable ribosomes 650, distinct plasma membrane 660, and a region of cell wall and fibrous extracellular layer 680 measuring 18-21 nm thick (FIGS. 6C-F). Samples prepared by passive diffusion under ambient or cryo conditions required approximately 2 and 5 days to process and infiltrate with resin, respectively. Microwave-assisted processing reduced that time period to less than 4 hours for both ambient and cryo-samples. A comparison of cryo-samples that were incubated for equal time periods with and without microwave irradiation showed that fixation was facilitated by the treatment (FIG. 6D (no microwave irradiation) and FIGS. 6E-F (with microwave irradiation).

The results shown in FIG. 6 demonstrate that MWFS produces comparable if not superior preservation and representation of B. subtilis ultrastructure versus that observed in samples prepared by conventional chemical fixation and ambient MW processing. The ultrastructure observed with MWFS was similar to that seen with passive FS with marked improvement in plasma membrane and cell wall preservation. Similarly, the structure observed with FS was consistent with previously published studies of B. subtilis (Grahamm, L. L., and Beveridge, T. J., J. Bacteriol. 176: 1413-21 (1994); Matias, V. R. F., and Beveridge, T. J., Mol. Microbiol. 56: 240-51 (2006). MW irradiation was required to achieve these results since samples incubated on dry ice in parallel with MWFS samples, but without MW treatment were not preserved sufficiently.

### Example 17

### Immunoelectron Microscopy of Membrane Protein in Intracellular Malaria Merozoites

This Example illustrates the immunoelectron microscopy of a membrane protein in intracellular malaria merozoite sections prepared using microwave-assisted cryo-sample processing. Plasmodium falciparum schizont-infected erythrocytes were fixed overnight at 4° C. with 0.075% glutaraldehyde/4% paraformaldehyde. The fixed erythrocytes were suspended in Hanks buffered saline solution with 10% BSA. The suspended erythrocytes were aliquoted to “hats” (Leica Microsystems, Vienna, Austria) in 1.5 μl aliquots for cryo-immobilization in a Leica EMPact2™.

Freeze substitution of the erythrocyte samples was performed with microwave irradiation. The samples were substituted with 1% uranyl acetate/0.1% glutaraldehyde in acetone and dehydrated using acetone within a Pelco 3451 microwave processor (Ted Pella, Redding, Calif.). During substitution and dehydration, the samples were maintained in crushed dry ice (approx. −85 to −65° C.). The microwave protocol for freeze substitution was as follows: 8 cycles of: 2 min on, 2 min off, 2 min. After substitution and dehydration, the samples were embedded in LR white resin. Thin sections were cut using an MT-7000 ultramicrotome (Ventana, Tucson, Ariz.), etched with 4% meta-periodate, and immunolabeled in a Pelco 3451 microwave oven using a Pelco PFTE immunostaining pad.

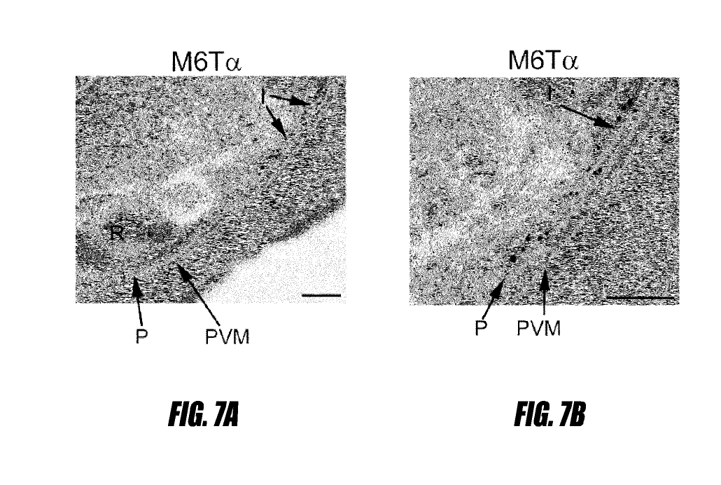

After the samples were blocked with 1% BSA/0.1% Tween 20 Tris buffer for 2 min at 150 Watts at 24° C. (additional steps retained same settings), the samples were incubated with primary antibody (anti-PfM6Tα antibody (Rayavara, K. et al., unpublished) for 2×2 min, washed 3×1 min and incubated with secondary 5 nm colloidal gold (BBInternational, Cardiff, UK) for 2×2 min before final rinsing. Sections were stained with 1% uranyl acetate and viewed on a Philips CM-10 TEM (FEI, Hillsboro, Oreg.) at 80 kV. Images were acquired with a Hammamatsu XR-100 digital camera system (Advanced Microscopy Techniques, Inc., Danvers, Mass.) (FIGS. 7A-B). Immuno-EM showed localization of PfM6Tα to the merozoite inner membrane complex (I), as indicated by electron dense colloidal gold particles (FIG. 7A). FIG. 7B is a subsection of FIG. 7A at higher magnification showing the gold particles in greater detail. Abbreviations: inner membrane complex (I), merozoite plasma membrane (P), rhoptry (R), parasitophorous vacuole membrane (PVM). Scale bars: 100 nm). The results shown in FIG. 7 demonstrate that MWFS produces excellent preservation and representation of merozoite membrane ultrastructure. Furthermore, the results show that the epitope recognized by the anti-PfM6Tα antibody remains intact throughout the MWFS procedure.

### Example 18

### Pre-Fixed Human Hela Cells, Processed by MWFS Using Sequential Treatment with Multiple Fixatives and Stains

This Example illustrates the usefulness of MWFS on human cells using sequential treatment with multiple fixatives and stains. Because of the extended time periods required and difficulty handling samples and reagents at cryo temperatures, protocols for diffusive freeze substitution involving multiple reagents have traditionally utilized mixtures of fixatives and stains in a single solvent rather than treating samples sequentially with multiple preparations containing single reagent/solvent mixtures. Such potentially complex mixtures can present problems due to variable properties including solubility in the mixture, reactivity between reagents in the mixture, potential deleterious effects on sample pH, and the like. However, development of automated instruments for MWFS, as proposed in this application, allow for precise temperature control of the sample and reagents, and the automated exchange of solutions. Accordingly, optimal reagent and solvent concentration and temperature for particular samples and applications are obtained, without having limitations on parameters due to mixture complexity or incompatibility. In order to assess the feasibility of such sequential MWFS processing, it was determined whether prefixed human HeLa cells could be frozen and processed rapidly using this system. Pre-fixed cells were chosen because many samples from laboratory and clinical facilities are biohazardous requiring thorough inactivation before freezing and handling.

In this experiment, chemical fixation with paraformaldehyde and a mixture of glutaraldehyde and malachite green were high-pressure frozen using the Leica EMPact2™ and processed further using sequential MWFS with osmium tetroxide, tannic acid, and uranyl acetate, as shown in Table 4.

TABLE 4

| | Power | | Temperature |
|---|---|---|---|
| Step | (W) | Time | (° C.) |
| 1% osmium | 250 | 8 cycles 2 min on, | crushed dry |
| tetroxide/acetone | | 2 min off, 2 min on | ice |
| | | | (approx. −85 |
| | | | to −65° C.) |
| Acetone, 3 changes | 250 | 45 sec each | crushed dry |
| | | | ice |
| | | | (approx. −85 |
| | | | to −65° C.) |
| 1% tannic acid/acetone | 250 | 8 cycles 2 min on, | crushed dry |
| | | 2 min off, 2 min on | ice |
| | | | (approx. −85 |
| | | | to −65° C.) |
| Acetone, 3 changes | 250 | 45 sec each | crushed dry |
| | | | ice |
| | | | (approx. −85 |
| | | | to −65° C.) |
| 1% uranyl | 250 | 8 cycles 2 min on, | crushed dry |
| acetate/acetone | | 2 min off, 2 min on | ice |
| | | | (approx. −85 |
| | | | to −65° C.) |
| Acetone, 3 changes | 250 | 45 sec each | crushed dry |
| | | | ice |
| | | | (approx. −85 |
| | | | to −65° C.) |
| Spurr's resin | 250 | 15 min on, 5 min off, | Ambient |
| | | 5 min on, and | (approx. |
| | | 90 min off | 24-30° C.) |

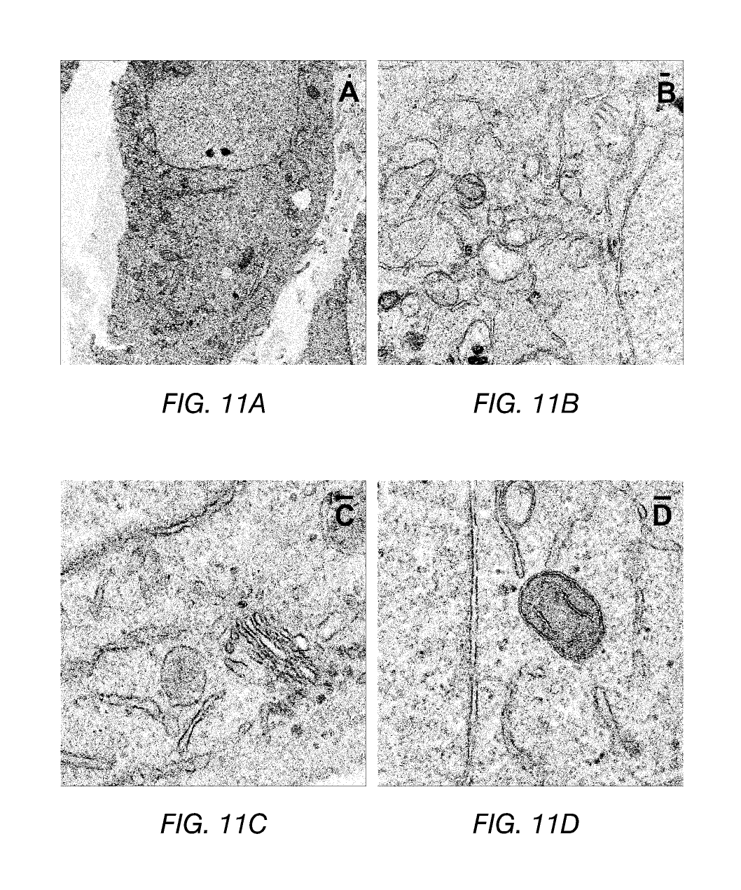

Each reagent was dissolved directly in acetone, and was removed by multiple acetone washes in between each step. It should be noted that the Spurr's resin was infiltrated into the cells under vacuum (˜250-500 Torr). The results are shown in FIG. 11A-D. The low magnification images shown in panels A and B show that the sequential fixation methods preserved morphology evenly and consistently throughout the material. Higher magnification panels C and D show that the fine structure of membranous organelles such as Golgi bodies (C) and mitochondria (D) were also well preserved. Thus, MWFS provides excellent preservation using sequential fixation protocols that can be accomplished in several hours by MWFS, as opposed to several days by diffusive freeze substitution. These results also provide strong evidence that instrumentation with automated reagent exchange along with MWFS, will enable development, optimization, and implementation of many protocols to maximize preservation and throughput of clinical and laboratory samples for light and electron microscopy.

### Example 19

### Comparison of Conventional Sample Preparation and MWFS Preparation of Plasmodium falciparum-Infected Human Red Blood Cells

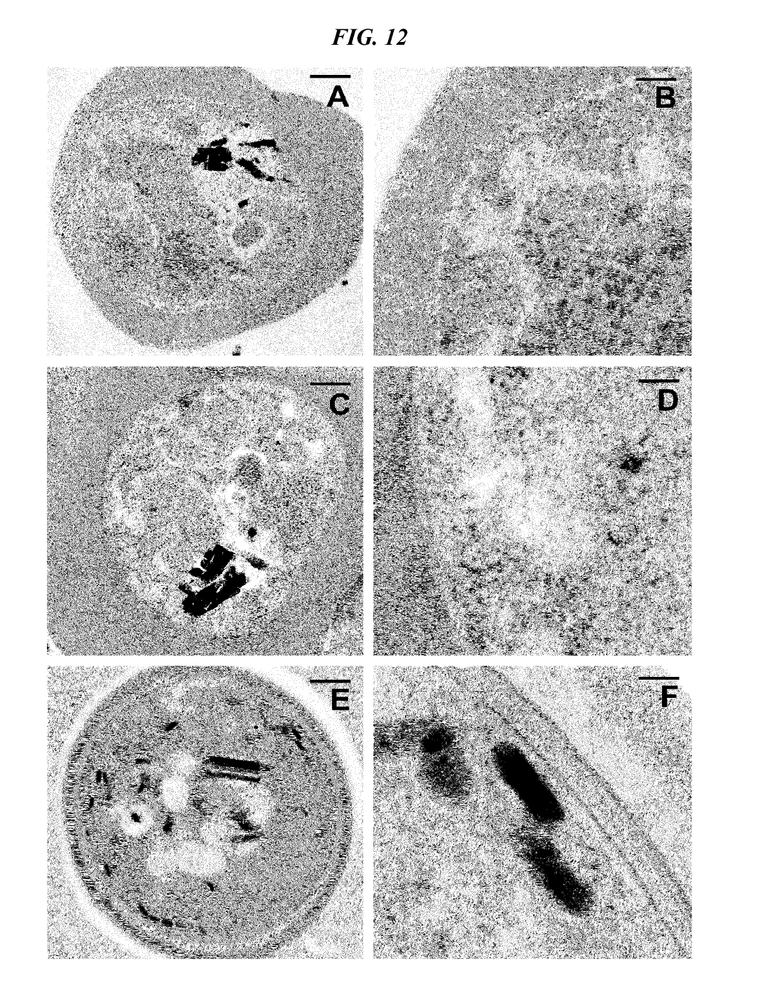

In this experiment, conventional microwave-assisted chemical fixation, traditional freeze substitution, and MWFS were used to process Plasmodium falciparum-infected human red blood cells (FIG. 12). As in Example 17, the samples were prefixed to inactivate the parasites before handling or freezing. Sample processing through post-fixation and infiltration required about an hour for microwave-assisted chemical fixation and MWFS. Samples prepared by traditional freeze substitution required approximately 2 days. Electron microscopy of sections prepared by each method showed that overall morphology and detailed fine structural preservation was significantly improved using MWFS. For example, whereas the parasite membrane complex was not preserved by ambient processing (FIG. 12 (A) and (B), and only poorly retained by traditional freeze substitution (FIG. 12 (C) and (D), sections prepared by MWFS clearly resolved multiple layers of membrane and intervening regions characteristic of the membrane complex (FIG. 12 (E) and (F).

While the present teachings have been described in terms of these exemplary embodiments, the skilled artisan will readily understand that numerous variations and modifications of these exemplary embodiments are possible without undue experimentation. All such variations and modifications are within the scope of the current teachings.

Although the disclosed teachings have been described with reference to various applications, methods, kits, and compositions, it will be appreciated that various changes and modifications can be made without departing from the teachings herein and the claimed invention below. The foregoing examples are provided to better illustrate the disclosed teachings and are not intended to limit the scope of the teachings presented herein.

In this application, the use of the singular can include the plural unless specifically stated otherwise or unless, as will be understood by one of skill in the art in light of the present disclosure, the singular is the only functional embodiment. Thus, for example, “a” can mean more than one, and “one embodiment” can mean that the description applies to multiple embodiments. Additionally, in this application, “and/or” denotes that both the inclusive meaning of “and” and, alternatively, the exclusive meaning of “or” applies to the list. Thus, the listing should be read to include all possible combinations of the items of the list and to also include each item, exclusively, from the other items. The addition of this term is not meant to denote any particular meaning to the use of the terms “and” or “or” alone. The meaning of such terms will be evident to one of skill in the art upon reading the particular disclosure.

All references cited herein, including patents, patent applications, papers, text books, and the like, and the references cited therein, to the extent that they are not already, are hereby incorporated by reference in their entirety. In the event that one or more of the incorporated literature and similar materials differs from or contradicts this application, including but not limited to defined terms, term usage, described techniques, or the like, this application controls.

The foregoing description and Examples detail certain specific embodiments of the invention and describes the best mode contemplated by the inventors. It will be appreciated, however, that no matter how detailed the foregoing may appear in text, the invention may be practiced in many ways and the invention should be construed in accordance with the appended claims and any equivalents thereof.

1. A system for microwave-assisted cryo-sample processing of a sample comprising:
a microwave generating device;
a sample chamber adapted to receive microwave radiation from said microwave generating device; and
a cooling/heating device in contact with the sample chamber such that the cooling/heating device is configured to maintain a sample in said sample chamber under cryo conditions during irradiation of the sample with microwave radiation.

2. The system of claim 1, wherein the cooling/heating device is adapted to conduct a cryogenic substance therethrough.

3. The system of claim 1, further comprising:
a sample holder comprising at least one well, wherein said well is configured to receive the sample, and wherein the sample holder is configured to be disposed in a recess in the cooling/heating device.

4. The system of claim 1, wherein the sample holder further comprises a temperature sensor.

5. The system of claim 1, wherein the cooling/heating device can be configured to maintain the temperature of the sample between about −200° C. and about 0° C.

6. The system of any claim 1, further comprising a temperature regulation system operably connected to the cooling/heating device.

7. The system of claim 1, further comprising a programmable controller.

8. The system of claim 7, wherein the controller can be programmed with a temperature setting.

9. The system of claim 1, further comprising a venting system for removing vapors from said sample.

10. The system of claim 1, further comprising a vacuum system for regulating sample pressure.

11. The system of claim 1, further comprising a dry-gas purge system for reducing moisture in said chamber.

12. The system of claim 1, further comprising a pressurized air system for regulating sample pressure.

13. The system of claim 1, wherein the microwave generating device comprises a magnetron.

14. The system of claim 1, wherein a chemical composition is disposed within the sample chamber, wherein said chemical composition is in contact with the sample.

15. The system of claim 14, configured such that the sample is substantially impregnated by the chemical composition in less than about two hours.

16. The system of claim 15, configured such that the sample is substantially impregnated by the chemical composition in less than about twenty minutes.

17. A cooling/heating device for microwave-assisted cryo-sample processing of a sample comprising:
a block comprising at least one opening sized to fit one or more samples, wherein the block is translucent or opaque to microwave irradiation and adapted to contain or conduct a cryogenic substance therethrough, and wherein a sample held by the block is maintained under cryo conditions during microwave irradiation.

18. The cooling/heating device of claim 17, further comprising a temperature sensor.

19.-28. (canceled)

29. A method for processing a sample for microscopy analysis comprising:
(a) irradiating a sample with a first power microwave radiation for a first set time, wherein the sample is maintained under cryo conditions and does not thaw during said first set time;
(b) irradiating the sample with a second power microwave radiation for a second set time;
(c) and contacting the sample with a first chemical composition during said first set time;
wherein the chemical composition selected from the group comprises acetone, methanol, or ethanol; wherein the chemical composition further comprises O_{S}O₄ (osmium tetroxide), uranyl acetate, tannic acid, glutaraldehyde, paraformaldehyde, formalin, ruthenium tetroxide, picric acid, malachite green, ruthenium red, alcian blue, potassium permanganate, or a carbodimide; wherein the same is selected from the group consisting of a bacterial and human cell.
